(19) United States
(12) Patent Application Publication
(10) Pub. No.: US 2020/0342979 A1
(43) Pub. Date: Oct. 29, 2020

(54) DISTRIBUTED ANALYSIS FOR COGNITIVE STATE METRICS

(71) Applicant: Affectiva, Inc., Boston, MA (US)

(72) Inventors: Richard Scott Sadowsky, Sturbridge, MA (US); Rana el Kaliouby, Milton, MA (US); Rosalind Wright Picard, Newtonville, MA (US); Oliver Orion Wilder-Smith, Holliston, MA (US); Panu James Turcot, Pacifica, CA (US); Zhihong Zheng, Lexington, MA (US)

(73) Assignee: Affectiva, Inc., Boston, MA (US)

(21) Appl. No.: 16/928,154

(22) Filed: Jul. 14, 2020

Related U.S. Application Data
(63) Continuation-in-part of application No. 16/900,026, filed on Jun. 12, 2020.
Application No. 16/900,026 is a continuation-in-part of application No. 16/017,037, filed on Jun. 25, 2018.
Application No. 16/017,037 is a continuation-in-part of application No. 14/328,554, filed on Jul. 10, 2014, now Pat. No. 10,111,611.
Application No. 14/328,554 is a continuation-in-part of application No. 13/153,745, filed on Jun. 6, 2011, now abandoned.
Application No. 16/900,026 is a continuation-in-part of application No. 14/068,919, filed on Oct. 31, 2013.
Application No. 14/068,919 is a continuation-in-part of application No. 13/708,214, filed on Dec. 7, 2012, now abandoned.
Application No. 14/068,919 is a continuation-in-part of application No. 13/153,745, filed on Jun. 6, 2011, now abandoned.
Application No. 13/153,745 is a continuation-in-part of application No. 15/382,087, filed on Dec. 16, 2016, now abandoned.
Application No. 15/382,087 is a continuation-in-part of application No. 15/262,197, filed on Sep. 12, 2016, now abandoned.
Application No. 15/262,197 is a continuation-in-part of application No. 14/796,419, filed on Jul. 10, 2015, now abandoned.
Application No. 14/796,419 is a continuation-in-part of application No. 14/460,915, filed on Aug. 15, 2014, now abandoned.
Application No. 14/460,915 is a continuation-in-part of application No. 13/153,745, filed on Jun. 6, 2011, now abandoned.
(60) Provisional application No. 62/955,493, filed on Dec. 31, 2019.
(60) Provisional application No. 62/954,819, filed on Dec. 30, 2019.
(60) Provisional application No. 62/954,833, filed on Dec. 30, 2019.
(60) Provisional application No. 62/925,990, filed on Oct. 25, 2019.
(60) Provisional application No. 62/926,009, filed on Oct. 25, 2019.
(60) Provisional application No. 62/893,298, filed on Aug. 29, 2019.
(60) Provisional application No. 62/679,825, filed on Jun. 3, 2018.
(60) Provisional application No. 62/637,567, filed on Mar. 2, 2018.
(60) Provisional application No. 62/625,274, filed on Feb. 1, 2018.
(60) Provisional application No. 62/611,780, filed on Dec. 29, 2017.
(60) Provisional application No. 62/593,440, filed on Dec. 1, 2017.
(60) Provisional application No. 62/593,449, filed on Dec. 1, 2017.
(60) Provisional application No. 62/557,460, filed on Sep. 12, 2017.
(60) Provisional application No. 62/541,847, filed on Aug. 7, 2017.
(60) Provisional application No. 62/524,606, filed on Jun. 25, 2017.
(60) Provisional application No. 61/927,481, filed on Jan. 15, 2014.
(60) Provisional application No. 61/924,252, filed on Jan. 7, 2014.
(60) Provisional application No. 61/916,190, filed on Dec. 14, 2013.
(60) Provisional application No. 61/844,478, filed on Jul. 10, 2013.
(60) Provisional application No. 61/467,209, filed on Mar. 24, 2011.
(60) Provisional application No. 61/447,464, filed on Feb. 28, 2011.
(60) Provisional application No. 61/447,089, filed on Feb. 27, 2011.
(60) Provisional application No. 61/439,913, filed on Feb. 6, 2011.
(60) Provisional application No. 61/414,451, filed on Nov. 17, 2010.
(60) Provisional application No. 61/388,002, filed on Sep. 30, 2010.
(60) Provisional application No. 61/352,166, filed on Jun. 7, 2010.
(60) Provisional application No. 61/844,478, filed on Jul. 10, 2013.
(60) Provisional application No. 61/789,038, filed on Mar. 15, 2013.
(60) Provisional application No. 61/790,461, filed on Mar. 15, 2013.
(60) Provisional application No. 61/793,761, filed on Mar. 15, 2013.
(60) Provisional application No. 61/798,731, filed on Mar. 15, 2013.
(60) Provisional application No. 61/747,651, filed on Dec. 31, 2012.
(60) Provisional application No. 61/747,810, filed on Dec. 31, 2012.
(60) Provisional application No. 61/581,913, filed on Dec. 30, 2011.
(60) Provisional application No. 61/568,130, filed on Dec. 7, 2011.
(60) Provisional application No. 62/370,421, filed on Aug. 3, 2016.
(60) Provisional application No. 62/301,558, filed on Feb. 29, 2016.
(60) Provisional application No. 62/273,896, filed on Dec. 31, 2015.
(60) Provisional application No. 62/265,937, filed on Dec. 10, 2015.
(60) Provisional application No. 62/222,518, filed on Sep. 23, 2015.
(60) Provisional application No. 62/217,872, filed on Sep. 12, 2015.
(60) Provisional application No. 62/128,974, filed on Mar. 5, 2015.
(60) Provisional application No. 62/082,579, filed on Nov. 20, 2014.
(60) Provisional application No. 62/047,508, filed on Sep. 8, 2014.
(60) Provisional application No. 62/023,800, filed on Jul. 11, 2014.
(60) Provisional application No. 61/972,314, filed on Mar. 30, 2014.
(60) Provisional application No. 61/953,878, filed on Mar. 16, 2014.
(60) Provisional application No. 61/927,481, filed on Jan. 15, 2014.
(60) Provisional application No. 61/924,252, filed on Jan. 7, 2014.
(60) Provisional application No. 61/916,190, filed on Dec. 14, 2013.
(60) Provisional application No. 61/867,007, filed on Aug. 16, 2013.

(51) Int. Cl. G16H 20/70; G06K 9/00; H04L 29/08; G16H 30/40; A61B 5/16; A61B 5/00

(52) U.S. Cl. CPC G16H 20/70; G06K 9/00315; G06K 9/00979; H04L 67/1097; A61B 5/015; A61B 5/165; A61B 5/0077; A61B 5/0022; A61B 5/0013; G16H 30/40

(57) ABSTRACT

Distributed analysis for cognitive state metrics is performed. Data for an individual is captured into a computing device. The data provides information for evaluating a cognitive state of the individual. The data for the individual is uploaded to a web server. A cognitive state metric for the individual is calculated. The cognitive state metric is based on the data that was uploaded. Analysis from the web server is received by the computing device. The analysis is based on the data for the individual and the cognitive state metric for the individual. An output that describes a cognitive state of the individual is rendered at the computing device. The output is based on the analysis that was received. The cognitive states of other individuals are correlated to the cognitive state of the individual. Other sources of information are aggregated. The information is used to analyze the cognitive state of the individual.

### RELATED APPLICATIONS

This application claims the benefit of U.S. provisional patent applications “Vehicle Interior Object Management” Ser. No. 62/893,298, filed Aug. 29, 2019, “Deep Learning In Situ Retraining” Ser. No. 62/925,990, filed Oct. 25, 2019, “Data Versioning for Neural Network Training” Ser. No. 62/926,009, filed Oct. 25, 2019, “Synthetic Data Augmentation for Neural Network Training” Ser. No. 62/954,819, filed Dec. 30, 2019, “Synthetic Data for Neural Network Training Using Vectors” Ser. No. 62/954,833, filed Dec. 30, 2019, and “Autonomous Vehicle Control Using Longitudinal Profile Generation” Ser. No. 62/955,493, filed Dec. 31, 2019.

This application is also a continuation-in-part of U.S. patent application “Media Manipulation Using Cognitive State Metric Analysis” Ser. No. 16/900,026, filed Jun. 12, 2020, which claims the benefit of U.S. provisional patent applications “Vehicle Interior Object Management” Ser. No. 62/893,298, filed Aug. 29, 2019, “Deep Learning In Situ Retraining” Ser. No. 62/925,990, filed Oct. 25, 2019, “Data Versioning for Neural Network Training” Ser. No. 62/926,009, filed Oct. 25, 2019, “Synthetic Data Augmentation for Neural Network Training” Ser. No. 62/954,819, filed Dec. 30, 2019, “Synthetic Data for Neural Network Training Using Vectors” Ser. No. 62/954,833, filed Dec. 30, 2019, and “Autonomous Vehicle Control Using Longitudinal Profile Generation” Ser. No. 62/955,493, filed Dec. 31, 2019.

The U.S. patent application “Media Manipulation Using Cognitive State Metric Analysis” Ser. No. 16/900,026, filed Jun. 12, 2020 is also a continuation-in-part of U.S. patent application “Image Analysis for Emotional Metric Generation” Ser. No. 16/017,037, filed Jun. 25, 2018, which claims the benefit of U.S. provisional patent applications “Image Analysis for Emotional Metric Generation” Ser. No. 62/524,606, filed Jun. 25, 2017, “Image Analysis and Representation for Emotional Metric Threshold Evaluation” Ser. No. 62/541,847, filed Aug. 7, 2017, “Multimodal Machine Learning for Emotion Metrics” Ser. No. 62/557,460, filed Sep. 12, 2017, “Speech Analysis for Cross-Language Mental State Identification” Ser. No. 62/593,449, filed Dec. 1, 2017, “Avatar Image Animation using Translation Vectors” Ser. No. 62/593,440, filed Dec. 1, 2017, “Directed Control Transfer for Autonomous Vehicles” Ser. No. 62/611,780, filed Dec. 29, 2017, “Cognitive State Vehicle Navigation Based on Image Processing” Ser. No. 62/625,274, filed Feb. 1, 2018, “Cognitive State Based Vehicle Manipulation Using Near Infrared Image Processing” Ser. No. 62/637,567, filed Mar. 2, 2018, and “Vehicle Manipulation Using Cognitive State” Ser. No. 62/679,825, filed Jun. 3, 2018.

The U.S. patent application “Image Analysis for Emotional Metric Generation” Ser. No. 16/017,037, filed Jun. 25, 2018 is also a continuation-in-part of U.S. patent application “Personal Emotional Profile Generation” Ser. No. 14/328,554, filed Jul. 11, 2014, which claims the benefit of U.S. provisional patent applications “Personal Emotional Profile Generation” Ser. No. 61/844,478, filed Jul. 10, 2013, “Heart Rate Variability Evaluation for Mental State Analysis” Ser. No. 61/916,190, filed Dec. 14, 2013, “Mental State Analysis Using an Application Programming Interface” Ser. No. 61/924,252, filed Jan. 7, 2014, and “Mental State Analysis for Norm Generation” Ser. No. 61/927,481, filed Jan. 15, 2014.

The U.S. patent application “Personal Emotional Profile Generation” Ser. No. 14/328,554, filed Jul. 11, 2014 is also a continuation-in-part of U.S. patent application “Mental State Analysis Using Web Services” Ser. No. 13/153,745, filed Jun. 6, 2011, which claims the benefit of U.S. provisional patent applications “Mental State Analysis Through Web Based Indexing” Ser. No. 61/352,166, filed Jun. 7, 2010, “Measuring Affective Data for Web-Enabled Applications” Ser. No. 61/388,002, filed Sep. 30, 2010, “Sharing Affect Data Across a Social Network” Ser. No. 61/414,451, filed Nov. 17, 2010, “Using Affect Within a Gaming Context” Ser. No. 61/439,913, filed Feb. 6, 2011, “Recommendation and Visualization of Affect Responses to Videos” Ser. No. 61/447,089, filed Feb. 27, 2011, “Video Ranking Based on Affect” Ser. No. 61/447,464, filed Feb. 28, 2011, and “Baseline Face Analysis” Ser. No. 61/467,209, filed Mar. 24, 2011.

The U.S. patent application “Media Manipulation Using Cognitive State Metric Analysis” Ser. No. 16/900,026, filed Jun. 12, 2020 is also a continuation-in-part of U.S. patent application “Optimizing Media Based on Mental State Analysis” Ser. No. 14/068,919, filed Oct. 31, 2013, which claims the benefit of U.S. provisional patent applications “Optimizing Media Based on Mental State Analysis” Ser. No. 61/747,651, filed Dec. 31, 2012, “Collection of Affect Data from Multiple Mobile Devices” Ser. No. 61/747,810, filed Dec. 31, 2012, “Mental State Analysis Using Heart Rate Collection Based on Video Imagery” Ser. No. 61/793,761, filed Mar. 15, 2013, “Mental State Data Tagging for Data Collected from Multiple Sources” Ser. No. 61/790,461, filed Mar. 15, 2013, “Mental State Analysis Using Blink Rate” Ser. No. 61/789,038, filed Mar. 15, 2013, “Mental State Well Being Monitoring” Ser. No. 61/798,731, filed Mar. 15, 2013, and “Personal Emotional Profile Generation” Ser. No. 61/844,478, filed Jul. 10, 2013.

The U.S. patent application “Optimizing Media Based on Mental State Analysis” Ser. No. 14/068,919, filed Oct. 31, 2013 is also a continuation-in-part of U.S. patent application “Mental State Analysis Using Web Services” Ser. No. 13/153,745, filed Jun. 6, 2011, which claims the benefit of U.S. provisional patent applications “Mental State Analysis Through Web Based Indexing” Ser. No. 61/352,166, filed Jun. 7, 2010, “Measuring Affective Data for Web-Enabled Applications” Ser. No. 61/388,002, filed Sep. 30, 2010, “Sharing Affect Data Across a Social Network” Ser. No. 61/414,451, filed Nov. 17, 2010, “Using Affect Within a Gaming Context” Ser. No. 61/439,913, filed Feb. 6, 2011, “Recommendation and Visualization of Affect Responses to Videos” Ser. No. 61/447,089, filed Feb. 27, 2011, “Video Ranking Based on Affect” Ser. No. 61/447,464, filed Feb. 28, 2011, and “Baseline Face Analysis” Ser. No. 61/467,209, filed Mar. 24, 2011.

The U.S. patent application “Optimizing Media Based on Mental State Analysis” Ser. No. 14/068,919, filed Oct. 31, 2013 is also a continuation-in-part of US patent application “Affect Based Evaluation of Advertisement Effectiveness” Ser. No. 13/708,214, filed Dec. 7, 2012, which claims the benefit of U.S. provisional patent applications “Mental State Evaluation Learning for Advertising” Ser. No. 61/568,130, filed Dec. 7, 2011 and “Affect Based Evaluation of Advertisement Effectiveness” Ser. No. 61/581,913, filed Dec. 30, 2011.

This application is also a continuation-in-part of U.S. patent application “Mental State Analysis Using Web Servers” Ser. No. 15/382,087, filed Mar. 16, 2018, which is a continuation-in-part of U.S. patent application “Mental State Analysis using Web Services” Ser. No. 13/153,745, filed Jun. 6, 2011, which claims the benefit of U.S. provisional patent applications “Mental State Analysis Through Web Based Indexing” Ser. No. 61/352,166, filed Jun. 7, 2010, “Measuring Affective Data for Web-Enabled Applications” Ser. No. 61/388,002, filed Sep. 30, 2010, “Sharing Affect Data Across a Social Network” Ser. No. 61/414,451, filed Nov. 17, 2010, “Using Affect Within a Gaming Context” Ser. No. 61/439,913, filed Feb. 6, 2011, “Recommendation and Visualization of Affect Responses to Videos” Ser. No. 61/447,089, filed Feb. 27, 2011, “Video Ranking Based on Affect” Ser. No. 61/447,464, filed Feb. 28, 2011, and “Baseline Face Analysis” Ser. No. 61/467,209, filed Mar. 24, 2011.

The U.S. patent application “Mental State Analysis Using Web Servers” Ser. No. 15/382,087, filed Mar. 16, 2018 is also a continuation-in-part of U.S. patent application “Mental State Event Signature Usage” Ser. No. 15/262,197, filed Sep. 12, 2016, which claims the benefit of U.S. provisional patent applications “Mental State Event Signature Usage” Ser. No. 62/217,872, filed Sep. 12, 2015, “Image Analysis In Support of Robotic Manipulation” Ser. No. 62/222,518, filed Sep. 23, 2015, “Analysis of Image Content with Associated Manipulation of Expression Presentation” Ser. No. 62/265,937, filed Dec. 10, 2015, “Image Analysis Using Sub-Sectional Component Evaluation To Augment Classifier Usage” Ser. No. 62/273,896, filed Dec. 31, 2015, “Analytics for Live Streaming Based on Image Analysis within a Shared Digital Environment” Ser. No. 62/301,558, filed Feb. 29, 2016, and “Deep Convolutional Neural Network Analysis of Images for Mental States” Ser. No. 62/370,421, filed Aug. 3, 2016.

The U.S. patent application “Mental State Event Signature Usage” Ser. No. 15/262,197, filed Sep. 12, 2016 is also a continuation-in-part of U.S. patent application “Mental State Event Definition Generation” Ser. No. 14/796,419, filed Jul. 10, 2015, which claims the benefit of U.S. provisional patent applications “Mental State Event Definition Generation” Ser. No. 62/023,800, filed Jul. 11, 2014, “Facial Tracking with Classifiers” Ser. No. 62/047,508, filed Sep. 8, 2014, “Semiconductor Based Mental State Analysis” Ser. No. 62/082,579, filed Nov. 20, 2014, and “Viewership Analysis Based On Facial Evaluation” Ser. No. 62/128,974, filed Mar. 5, 2015.

The U.S. patent application “Mental State Event Definition Generation” Ser. No. 14/796,419, filed Jul. 10, 2015 is also a continuation-in-part of U.S. patent application “Mental State Analysis Using Web Services” Ser. No. 13/153,745, filed Jun. 6, 2011, which claims the benefit of U.S. provisional patent applications “Mental State Analysis Through Web Based Indexing” Ser. No. 61/352,166, filed Jun. 7, 2010, “Measuring Affective Data for Web-Enabled Applications” Ser. No. 61/388,002, filed Sep. 30, 2010, “Sharing Affect Across a Social Network” Ser. No. 61/414,451, filed Nov. 17, 2010, “Using Affect Within a Gaming Context” Ser. No. 61/439,913, filed Feb. 6, 2011, “Recommendation and Visualization of Affect Responses to Videos” Ser. No. 61/447,089, filed Feb. 27, 2011, “Video Ranking Based on Affect” Ser. No. 61/447,464, filed Feb. 28, 2011, and “Baseline Face Analysis” Ser. No. 61/467,209, filed Mar. 24, 2011.

The U.S. patent application “Mental State Event Definition Generation” Ser. No. 14/796,419, filed Jul. 10, 2015 is also a continuation-in-part of U.S. patent application “Mental State Analysis Using an Application Programming Interface” Ser. No. 14/460,915, Aug. 15, 2014, which claims the benefit of U.S. provisional patent applications “Application Programming Interface for Mental State Analysis” Ser. No. 61/867,007, filed Aug. 16, 2013, “Mental State Analysis Using an Application Programming Interface” Ser. No. 61/924,252, filed Jan. 7, 2014, “Heart Rate Variability Evaluation for Mental State Analysis” Ser. No. 61/916,190, filed Dec. 14, 2013, “Mental State Analysis for Norm Generation” Ser. No. 61/927,481, filed Jan. 15, 2014, “Expression Analysis in Response to Mental State Express Request” Ser. No. 61/953,878, filed Mar. 16, 2014, “Background Analysis of Mental State Expressions” Ser. No. 61/972,314, filed Mar. 30, 2014, and “Mental State Event Definition Generation” Ser. No. 62/023,800, filed Jul. 11, 2014.

The U.S. patent application “Mental State Analysis Using an Application Programming Interface” Ser. No. 14/460,915, Aug. 15, 2014 is also a continuation-in-part of U.S. patent application “Mental State Analysis Using Web Services” Ser. No. 13/153,745, filed Jun. 6, 2011, which claims the benefit of U.S. provisional patent applications “Mental State Analysis Through Web Based Indexing” Ser. No. 61/352,166, filed Jun. 7, 2010, “Measuring Affective Data for Web-Enabled Applications” Ser. No. 61/388,002, filed Sep. 30, 2010, “Sharing Affect Across a Social Network” Ser. No. 61/414,451, filed Nov. 17, 2010, “Using Affect Within a Gaming Context” Ser. No. 61/439,913, filed Feb. 6, 2011, “Recommendation and Visualization of Affect Responses to Videos” Ser. No. 61/447,089, filed Feb. 27, 2011, “Video Ranking Based on Affect” Ser. No. 61/447,464, filed Feb. 28, 2011, and “Baseline Face Analysis” Ser. No. 61/467,209, filed Mar. 24, 2011.

Each of the foregoing applications is hereby incorporated by reference in its entirety.

### FIELD OF INVENTION

This application relates generally to distributed analysis and more particularly to distributed analysis for cognitive state metrics.

### BACKGROUND

As technology companies dealing with “big data” and high workloads grow, so do their computational needs. For example, a traditional database can start on a single machine. As data and traffic increase, the machine requires hardware upgrades to maintain performance. This is called vertical scaling. Eventually, even the best and most expensive hardware upgrades become insufficient. To manage increasing traffic and performance demands, companies have turned to horizontal scaling, which adds more computers instead of upgrading a single system. Distributed computing uses multiple autonomous computer systems to solve computational problems.

A problem can be divided into many tasks, and each is solved by one or more networked computers that communicate by passing messages. Multiple software components are located on multiple computers, but they operate as a single system. The distributed computing system can include mainframes, personal computers, workstations, servers, and minicomputers. The computers can be physically located close together, where they can be connected via a local network. If the computers are geographically distant, they can be connected by a Wide Area Network. Though multiple machines are working together to achieve a common goal, to the end user, the group of machines appears as a single computer. All distributed computing systems share several characteristics. The computers do not share a clock. The computers do not share memory. The software and hardware components are autonomous and complete tasks concurrently. The processors are separate, independent, and have their own speeds. It can be difficult to get separate, independent processors to work together efficiently.

### SUMMARY

Distributed analysis for cognitive state metrics is performed. Data for an individual is captured into a computing device. The data provides information for evaluating a cognitive state of the individual. The data for the individual is uploaded to a web server. A cognitive state metric for the individual is calculated. The cognitive state metric is based on the data that was uploaded. Analysis from the web server is received by the computing device. The analysis is based on the data for the individual and the cognitive state metric for the individual. An output that describes a cognitive state of the individual is rendered at the computing device. The output is based on the analysis that was received. The cognitive states of other individuals are correlated to the cognitive state of the individual. Other sources of information are aggregated. The information is used to analyze the cognitive state of the individual.

A computer-implemented method for distributed analysis is disclosed comprising: capturing data for an individual into a computing device, wherein the data provides information for evaluating a cognitive state of the individual; uploading the data for the individual to a web server; calculating a cognitive state metric for the individual, on the web server, based on the data that was uploaded; receiving analysis from the web server, by the computing device, wherein the analysis is based on the data for the individual and the cognitive state metric for the individual; and rendering an output at the computing device that describes a cognitive state of the individual, based on the analysis that was received. The cognitive state metric can be based on a facial expression metric for the individual. The facial expression metric for the individual can be calculated on facial image data captured as part of the data for the individual. The calculation on facial image data can be performed on the web server. The calculation on facial image data can be performed on the computing device before uploading to the web server. An emotional intensity metric can be included in the cognitive state metric

The cognitive state metric can include a cognitive state and an emotional state. Facial data included with the data for an individual can include information on facial expressions, action units, head gestures, smiles, squints, lowered eyebrows, raised eyebrows, smirks, and attention. The method can further comprise inferring cognitive states, based on the data that was collected and the analysis of the facial data. The web server can comprise an interface which includes a cloud-based server that is remote to the individual and cloud-based storage. The web server can comprise an interface which includes a datacenter-based server that is remote to the individual and datacenter-based storage. The method can further comprise indexing the data on the individual through the web server. The indexing can include categorization based on valence and arousal information. The method can further comprise receiving analysis information on a plurality of other individuals, wherein the analysis information allows evaluation of a collective cognitive state of the plurality of other individuals. The analysis information can include correlation for the cognitive state of the plurality of other individuals to the data that was captured on the cognitive state of the individual. The correlation can be based on metadata from the individual and metadata from the plurality of other people. The correlation can be based on the comparing of the data that was captured for the individual against a plurality of cognitive state event temporal signatures.

The analysis which is received from the web server can be based on specific access rights. The method can further comprise sending a request to the web server for the analysis. The analysis can be generated just in time based on the request for the analysis. The method can further comprise sending a subset of the data which was captured on the individual to the web server. The rendering can be based on data which is received from the web server. The data which is received can include a serialized object in a form of JavaScript Object Notation (JSON). The method can further comprise de-serializing the serialized object into a form for a JavaScript object. The rendering can further comprise recommending a course of action based on the cognitive state of the individual. The recommending can include modifying a question queried to a focus group, changing an advertisement on a web page, editing a movie which was viewed to remove an objectionable section, changing direction of an electronic game, changing a medical consultation presentation, or editing a confusing section of an internet-based tutorial.

Various features, aspects, and advantages of various embodiments will become more apparent from the following further description.

### BRIEF DESCRIPTION OF THE DRAWINGS

The following detailed description of certain embodiments can be understood by reference to the following figures wherein:

FIG. 1 is a flow diagram of distributed analysis for cognitive state metrics.

FIG. 2 is an example system diagram of distributed analysis for cognitive state metrics.

FIG. 3 is a graphical rendering of electrodermal activity.

FIG. 4 is a graphical rendering of accelerometer data.

FIG. 5 is a graphical rendering of skin temperature data.

FIG. 6 shows an image collection system for facial analysis.

FIG. 7 is a flow diagram for performing facial analysis.

FIG. 8 is a diagram describing physiological analysis.

FIG. 9 is a flow diagram describing heart rate analysis.

FIG. 10 is a flow diagram for performing cognitive state analysis and rendering.

FIG. 11 is a flow diagram describing analysis of the cognitive response of a group.

FIG. 12 is a flow diagram for identifying data portions which match a selected cognitive state of interest.

FIG. 13 is a graphical rendering of cognitive state analysis along with an aggregated result from a group of individuals.

FIG. 14 is a graphical rendering of cognitive state analysis.

FIG. 15 is a graphical rendering of cognitive state analysis based on metadata.

FIG. 16 is a flow diagram for cognitive state-based recommendations.

FIG. 17 shows example image collection including multiple mobile devices.

FIG. 18 is an example showing a pipeline for facial analysis layers.

FIG. 19 is an example illustrating a deep network for facial expression parsing.

FIG. 20 is an example illustrating a convolution neural network.

FIG. 21 is a system diagram for an interior of a vehicle.

FIG. 22 illustrates a bottleneck layer within a deep learning environment.

FIG. 23 shows data collection including multiple devices and locations.

FIG. 24A shows example tags embedded in a webpage.

FIG. 24B shows example invoking tags for the collection of images.

FIG. 25 shows an example livestreaming social video scenario.

FIG. 26 is a system diagram for cognitive state metric analysis.

### DETAILED DESCRIPTION

The present disclosure provides a description of various methods and systems for distributed analysis for cognitive state metrics. A metric is a quantitative approach to providing an objective measure of a cognitive state or an emotional state, which can be broadly covered using the term affect. Examples of emotional states include happiness or sadness. Examples of cognitive states include concentration or confusion. Observing, capturing, and analyzing these cognitive states can yield significant information about people's reactions to various stimuli. Some terms commonly used in the evaluation of cognitive states are arousal and valence. Arousal is an indication on the amount of activation or excitement of a person. Valence is an indication on whether a person is positively or negatively disposed. Determination of affect can include analysis of arousal and valence. Determining affect can also include facial analysis for expressions such as smiles or brow furrowing. Analysis can be as simple as tracking when someone smiles or when someone frowns. Beyond this, recommendations for courses of action can be made based on tracking when someone smiles or demonstrates another affect.

The present disclosure provides a description of various methods and systems associated with distributed analysis for cognitive state metrics. Emotional state, cognitive state, mental state, affect, and so on, are terms of art which may connote slight differences of emphasis, for example an emotional state of “happiness” vs. a cognitive state of “distractedness,” but at a high level, the terms can be used interchangeably. In fact, because the human mind of an individual is often difficult to understand—even for the individual—emotional, mental, and cognitive states may easily be overlapping and appropriately used interchangeably in a general sense.

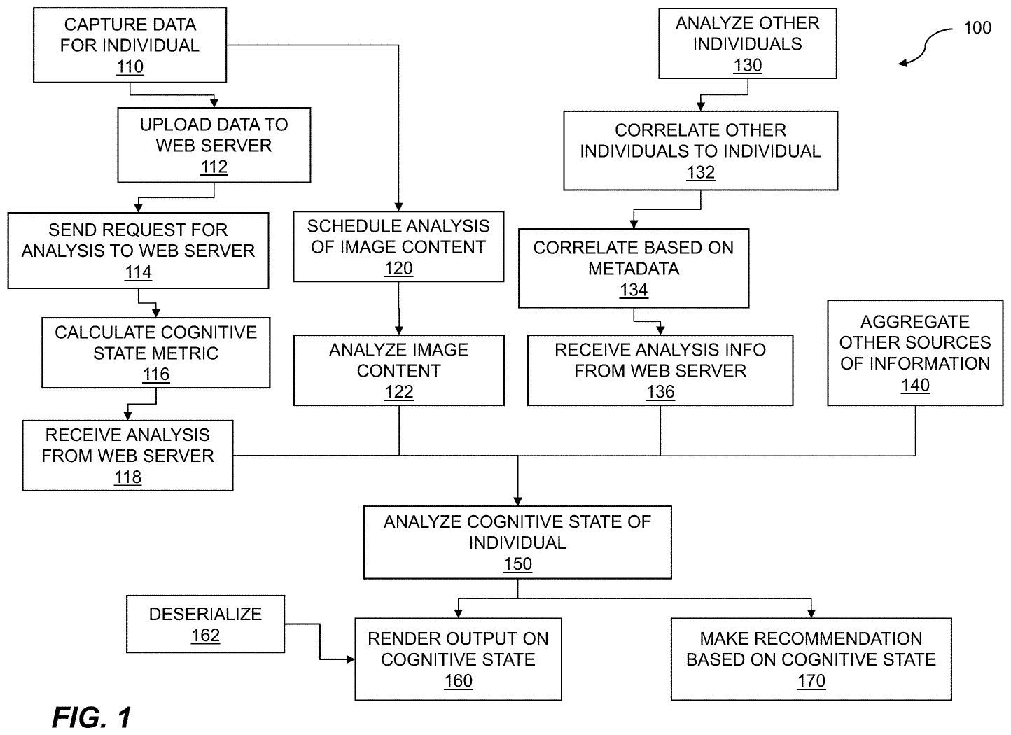

FIG. 1 is a flow diagram of distributed analysis for cognitive state metrics. The flow 100 describes a computer-implemented method for distributed analysis for cognitive state metrics. The flow begins by capturing data for an individual 110 into a computer system, wherein the data provides information for evaluating the cognitive state of the individual. The data which was captured can be correlated to an experience by the individual. The experience can comprise interacting with a website, a movie, a movie trailer, a product, a computer game, a video game, a personal game console, a cell phone, a mobile device, an advertisement. The experience can further include consuming a food. “Interacting with” can refer to simply viewing, or it can mean viewing and responding. The data on the individual can further include information on hand gestures and body language. The data on the individual can include facial expressions, physiological information, and accelerometer readings. The facial expressions can further comprise head gestures. The physiological information can include electrodermal activity, skin temperature, heart rate, heart rate variability, and respiration. The physiological information can be obtained without physically contacting the individual, such as through analyzing facial video. The information can be captured and analyzed in real time, on a just-in-time basis, or on a scheduled analysis basis.

The flow 100 continues with uploading the data that was captured to a web server 112. The sent data can include image, physiological, and accelerometer information. The data can be sent for cognitive state analysis or for correlation with other people's data or another analysis. In some embodiments, the data which is sent to the web service is a subset of the data that was captured on the individual. The web servers can be a website, File Transfer Protocol (FTP) site, or server which provides access to a larger group of analytical tools and data relating to cognitive states. The web servers can provide a conduit for data that was collected on other people or from other sources of information. In some embodiments, the process includes indexing the data which was captured on a web service. The flow 100 can continue with sending a request for analysis to the web server 114. The analysis can include correlating the data which was captured with other people's data, analyzing the data which was captured for cognitive states, and the like. The analysis can include calculating a cognitive state metric 116 for the data. The cognitive state metric can include a quantitative, objective measurement of the cognitive state. For example, a cognitive state may be “happy,” but a cognitive state metric for happiness may include an integer between 0 and 100, where a metric score near 100 indicates a high degree of happiness and a metric score near 0 indicates a low degree of happiness. In some embodiments, the analysis is generated just-in-time based on a request for the analysis. The flow 100 continues with receiving analysis from the web server 118. The analysis can be based on the data for the individual which was captured. The received analysis can correspond to what was requested, can be based on the data captured, or can be some other logical analysis based on the cognitive state analysis or the data that was captured recently.

In some embodiments, the data which was captured includes images of the individual. The images can be a sequence of images and can be captured by video camera, web camera still shots, thermal imager, CCD devices, phone camera, or another camera type apparatus. The flow 100 can include scheduling analysis of the image content 120. The analysis can be performed in real time, on a just-in-time basis, or scheduled for later analysis. Some of the data that was captured can require further analysis beyond what is possible in real time. Other types of data can also require further analysis and can involve scheduling analysis of a portion of the data which was captured and indexed and performing the analysis of the portion of the data which was scheduled. The flow 100 can continue with analysis of the image content 122. In some embodiments, analysis of video includes the data on facial expressions and head gestures. The facial expressions and head gestures can be recorded on video. The video can be analyzed for action units, gestures, and cognitive states. In some embodiments, the video analysis is used to evaluate skin pore size, which can be correlated to skin conductance or another physiological evaluation. In some embodiments, the video analysis is used to evaluate pupil dilation.

The flow 100 includes analyzing other individuals 130. Information from a plurality of other individuals can be analyzed, wherein the information allows evaluation of the cognitive state of each of the plurality of other individuals and correlates the cognitive state of each of the plurality of other individuals to the data which was captured and indexed on the cognitive state of the individual. Evaluation for a collective cognitive state of the plurality of other individuals can also be allowed. The other individuals can be grouped based on demographics, based on geographical locations, or based on other factors of interest in the evaluation of cognitive states. The analysis can include each type of data captured for the individual 110. Alternatively, analysis on the other individuals 130 can include other data, such as social media network information. The other individuals, and their associated data, can be correlated to the individual 132 on which the data was captured. The correlation can be based on common experiences, common cognitive states, common demographics, or other factors. In some embodiments, the correlation is based on metadata 134 from the individual and metadata from the plurality of other people. The metadata can include time stamps, self-reporting results, and other information. Self-reporting results can include an indication of whether someone liked the experience they encountered, such as a video that was viewed. The flow 100 can continue with receiving analysis information from the web server 136 on the plurality of other individuals, wherein the information allows evaluation of the cognitive state of each of the plurality of other individuals and correlation of the cognitive state of each of the plurality of other individuals to the cognitive state data that was captured for the individual. The analysis which is received from the web server or web service can be based on specific access rights. A web service can have data on numerous groups of individuals. In some cases, cognitive state analysis can be authorized on only one or more certain groups.

The flow 100 can include aggregating other sources of information 140 in the cognitive state analysis effort. The sources of information can include newsfeeds, Facebook™ entries, Flickr™, Twitter™ tweets, and other social networking sites. The aggregating can involve collecting information from the various sites which the individual visits or for which the individual creates content. The other sources of information can be correlated to the individual to help determine the relationship between the individual's cognitive states and the other sources of information.

The flow 100 continues with analysis of the cognitive states of the individual 150. The data which was captured, the image content which was analyzed, the correlation to the other people, and the other sources of information which were aggregated can each be used to infer one or more cognitive states for the individual. The data can be analyzed to produce cognitive state information. Further, a cognitive state analysis can be performed for a group of people, including the individual and one or more people from the other people. The process can include automatically inferring a cognitive state based on the data on the individual that was captured. The cognitive state can be a cognitive state, an emotional state, or a combination of cognitive and affective states. A cognitive state can be inferred, or a cognitive state can be estimated along with a probability for the individual experiencing that cognitive state. The cognitive state can be expressed as a cognitive state metric rather than a qualitative description. The cognitive states that can be evaluated can include happiness, sadness, contentedness, worry, concentration, anxiety, confusion, delight, and confidence. In some embodiments, an indicator of cognitive state is simply tracking and analyzing smiles.

Cognitive states can be inferred based on physiological data, on accelerometer readings, or on facial images which are captured. The cognitive states can be analyzed based on arousal and valence. Arousal can range from being highly activated, such as when someone is agitated, to being entirely passive, such as when someone is bored. Valence can range from being very positive, such as when someone is happy, to being very negative, such as when someone is angry. Physiological data can include electrodermal activity (EDA) or skin conductance or galvanic skin response (GSR), accelerometer readings, skin temperature, heart rate, heart rate variability, and other types of analysis of a human being. It will be understood that both here and elsewhere in this document, physiological information can be obtained either by sensor or by facial observation. In some embodiments, the facial observations are obtained with a webcam. In some instances, an elevated heart rate indicates a state of excitement. An increased level of skin conductance can correspond to being aroused. Small, frequent accelerometer movement readings can indicate fidgeting and boredom. Accelerometer readings can also be used to infer context, such as working at a computer, riding a bicycle, or playing a guitar. Facial data can include facial actions and head gestures used to infer cognitive states. Further, the data can include information on hand gestures or body language and body movements such as visible fidgets. In some embodiments, these movements are captured by cameras or sensor readings. Facial data can include tilting the head to the side, leaning forward, smiling, frowning, and many other gestures or expressions. Tilting of the head forward can indicate engagement with what is being shown on an electronic display. Having a furrowed brow can indicate concentration. A smile can indicate being positively disposed or being happy. Laughing can indicate that a subject has been found to be funny and enjoyable. A tilt of the head to the side and a furrow of the brows can indicate confusion. A shake of the head negatively can indicate displeasure. These and many other cognitive states can be indicated based on facial expressions and physiological data that is captured. In embodiments, physiological data, accelerometer readings, and facial data are each used as contributing factors in algorithms that infer various cognitive states. Additionally, higher complexity cognitive states can be inferred from multiple pieces of physiological data, facial expressions, and accelerometer readings. Further, cognitive states can be inferred based on physiological data, facial expressions, and accelerometer readings collected over a period of time.

The flow 100 continues with rendering an output that describes the cognitive state 160 of the individual based on the analysis which was received. The output can be a textual or numeric output indicating one or more cognitive states. The output can be a graph with a timeline of an experience and the cognitive states encountered during that experience. The output rendered can be a graphical representation of physiological, facial, or accelerometer data collected. Likewise, a result can be rendered which shows a cognitive state and the probability of the individual experiencing that cognitive state. The process can include annotating the data which was captured and rendering the annotations. The rendering can display the output on a computer screen. The rendering can include displaying arousal and valence. The rendering can store the output on a computer readable memory in the form of a file or data within a file. The rendering can be based on data which is received from the web service. Various types of data can be received, including a serialized object in the form of JavaScript Object Notation (JSON) or in an XML or CSV type file. The flow 100 can include deserializing 162 the serialized object into a form for a JavaScript object. The JavaScript object can then be used to output text or graphical representations of the cognitive states.

In some embodiments, the flow 100 includes recommending a course of action based on the cognitive state 170 of the individual. The recommending can include modifying a question queried to a focus group, changing an advertisement on a web page, editing a movie which was viewed to remove an objectionable section, changing direction of an electronic game, changing a medical consultation presentation, editing a confusing section of an internet-based tutorial, or the like. Various steps in the flow 100 may be changed in order, repeated, omitted, or the like without departing from the disclosed concepts. Various embodiments of the flow 100 may be included in a computer program product embodied in a non-transitory computer readable medium that includes code executable by one or more processors.

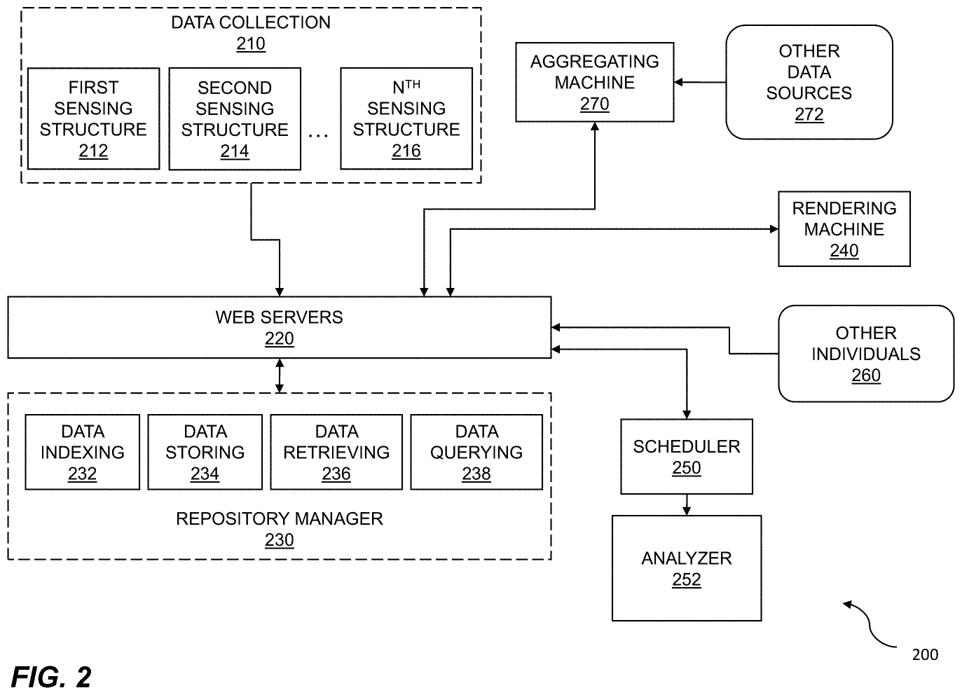

FIG. 2 is an example system diagram of distributed analysis for cognitive state metrics. The system 200 can include data collection 210, web servers 220, a repository manager 230, an analyzer 252, and a rendering machine 240. The data collection 210 can be accomplished by collecting data from a plurality of sensing structures, such as a first sensing structure 212, a second sensing structure 214, through an n^{th} sensing structure 216. This plurality of sensing structures can be attached to an individual, be near to the individual, or can view the individual. These sensing structures can be adapted to perform facial analysis. The sensing structures can be adapted to perform physiological analysis which can include electrodermal activity or skin conductance, accelerometer data, skin temperature, heart rate, heart rate variability, respiration, and other types of analysis of a human being. The data collected from these sensing structures can be analyzed in real time or can be collected for later analysis, based on the processing requirements of the needed analysis. The analysis can also be performed “just in time.” A just-in-time analysis can be performed on request, where the result is provided when a button is clicked on in a web page, for instance. Analysis can also be performed as data is collected so that a timeline, with associated analysis, is presented in real time while the data is being collected with little or no time lag from the collection. In this manner, the analysis results can be presented while data is still being collected on the individual.

The web servers 220 can comprise an interface which includes a server that is remote to the individual and cloud-based storage. Web servers can include a website, FTP site, or server which provides access to a larger group of analytical tools for cognitive states. The web servers 220 can also be a conduit for data that was collected as it is routed to other parts of the system 200. The web servers 220 can be a server or a distributed network of computers. The web servers 220 can be cloud based. The web servers 220 can be datacenter based. The datacenter-based web server can be remote from the individual and can include datacenter-based storage. The web servers 220 can provide a means for a user to log in and request information and analysis. The information request can take the form of analyzing a cognitive state for an individual in light of various other sources of information or based on a group of people which correlate to the cognitive state for the individual of interest. In some embodiments, the web servers 220 provide for forwarding data which was collected to one or more processors for further analysis.

The web servers 220 can forward the data which was collected to a repository manager 230. The repository manager can provide for data indexing 232, data storing 234, data retrieving 236, and data querying 238. The data which was collected through the data collection 210, through, for example, a first sensing structure 212, can be forwarded through the web servers 220 to the repository manager 230. The repository manager can, in turn, store the data which was collected. The data on the individual can be indexed, through web servers, with other data that has been collected for the individual on which the data collection 210 has occurred or can be indexed with other individuals whose data has been stored in the repository manager 230. The indexing can include categorization based on valence and arousal information. The indexing can include ordering based on time stamps or other metadata. The indexing can include correlating the data based on common cognitive states or based on a common experience of individuals. The common experience can be viewing or interacting with a website, a movie, a movie trailer, an advertisement, a television show, a streamed video clip, a distance learning program, a video game, a computer game, a personal game machine, a cell phone, an automobile or another vehicle, a product, a web page; consuming a food; and so forth. Other experiences for which cognitive states can be evaluated include walking through a store or a shopping mall, or encountering a display within a store.

Multiple types of indexing can be performed. The data, such as facial expressions or physiological information, can be indexed. One type of index can be a tightly bound index where a clear relationship exists, which might be useful in future analysis. One example is time stamping of the data in hours, minutes, seconds, and perhaps, in certain cases, fractions of a second. Other examples include a project, client, or individual being associated with data. Another type of index can be a looser coupling, where certain possibly useful associations might not be self-evident at the start of an effort. Some examples of these types of indexing include employment history, gender, income, or other metadata. Another example is the location where the data was captured, for instance in the individual's home, workplace, school, or another setting. Yet another example includes information on the person's action or behavior. Instances of this type information include whether a person performed a check-out operation while on a website, whether they completed certain forms, what queries or searches they performed, and the like. The time of day when the data was captured might prove useful for some types of indexing, as might be the work shift time when the individual normally works. Any sort of information which might be indexed can be collected as metadata. Indices can be formed in an ad hoc manner and retained temporarily while certain analysis is performed. Alternatively, indices can be formed and stored with the data for future reference. Further, metadata can include self-report information from the individuals on which data is collected.

Data can be retrieved through accessing the web servers 220 and requesting data which was collected for an individual. Data can also be retrieved for a collection of individuals, for a given time period, or for a given experience. Data can be queried to find matches for a specific experience, for a given mental response or cognitive state, or for an individual or group of individuals. Associations can be found through queries and various retrievals which might prove useful in a business or therapeutic environment. Queries can be made based on key word searches, a time frame, or an experience.

In some embodiments, a display is provided using a rendering machine 240. The rendering machine 240 can be part of a computer system which is part of another component of the system 200, part of the web servers 220, or part of a client computer system. The rendering can include graphical display of information collected in the data collection 210. The rendering can include display of video, electrodermal activity, accelerometer readings, skin temperature, heart rate, and heart rate variability. The rendering can also include display of cognitive states. In some embodiments, the rendering includes probabilities of certain cognitive states. The cognitive state for the individual can be inferred based on the data which was collected and can be based on facial analysis of activity units as well as facial expressions and head gestures. For instance, concentration can be identified by a furrowing of eyebrows. An elevated heart rate can indicate being excited. Reduced skin conductance can correspond to arousal. These and other factors can be used to identify cognitive states which might be rendered in a graphical display.

The system 200 can include a scheduler 250. The scheduler 250 can obtain data that came from the data collection 210. The scheduler 250 can interact with an analyzer 252. The scheduler 250 can determine a schedule for analysis by the analyzer 252 where the analyzer 252 is limited by computer processing capabilities where the data cannot be analyzed in real time. In some embodiments, aspects of the data collection 210, the web servers 220, the repository manager 230, or other components of the system 200 require computer processing capabilities for which the analyzer 252 is used. The analyzer 252 can be a single processor, multiple processors, or a networked group of processors. The analyzer 252 can include various other computer components, such as memory and the like, to assist in performing the needed calculations for the system 200. The analyzer 252 can communicate with the other components of the system 200 through the web servers 220. In some embodiments, the analyzer 252 communicates directly with the other components of the system. The analyzer 252 can provide an analysis result for the data which was collected from the individual, wherein the analysis result is related to the cognitive state of the individual. In some embodiments, the analyzer 252 provides results on a just-in-time basis. The scheduler 250 can request just-in-time analysis by the analyzer 252.

Information from other individuals 260 can be provided to the system 200. The other individuals 260 can have a common experience with the individual on which the data collection 210 was performed. The process can include analyzing information from a plurality of other individuals 260, wherein the information allows evaluation of the cognitive state of each of the plurality of other individuals 260, and correlating the cognitive state of each of the plurality of other individuals 260 to the data which was captured and indexed on the cognitive state of the individual. Metadata can be collected on each of the other individuals 260 or on the data collected on the other individuals 260. Alternatively, the other individuals 260 can have a correlation for cognitive states with the cognitive state for the individual on which the data was collected. The analyzer 252 can further provide a second analysis based on a group of other individuals 260, wherein cognitive states for the other individuals 260 correlate to the cognitive state of the individual. In other embodiments, a group of other individuals 260 is analyzed with the individual on whom data collection was performed to infer a cognitive state that represents a response of the entire group and is referred to as a collective cognitive state. This response can be used to evaluate the value of an advertisement, the likeability of a political candidate, how enjoyable a movie is, and so on. Analysis can be performed on the other individuals 260 so that collective cognitive states of the overall group can be summarized. The rendering can include displaying collective cognitive states from the plurality of individuals.

For example, a hundred people can view several movie trailers, with facial and physiological data captured from each. The facial and physiological data can be analyzed to infer the cognitive states of each individual and the collective response of the group as a whole. The movie trailer which has the greatest arousal and positive valence can be considered to motivate viewers of the movie trailer to be positively predisposed to go see the movie when it is released. Based on the collective response, the best movie trailer can then be selected for use in advertising an upcoming movie. In some embodiments, the demographics of the individuals are used to determine which movie trailer is best suited for different viewers. For example, one movie trailer can be recommended where teenagers will be the primary audience. Another movie trailer can be recommended where the parents of the teenagers will be the primary audience. In some embodiments, webcams or other cameras are used to analyze the gender and age of people as they interact with media. Further, IP addresses can be collected indicating the geographical location where analysis is being collected. This information and other information can be included as metadata and can be used as part of the analysis. For instance, teens who are up past midnight on Friday nights in an urban setting might be identified as a group for analysis.

In another example, a dozen individuals can opt in for allowing web cameras to observe facial expressions and then have physiological responses collected while they are interacting with a website for a given retailer. The cognitive states of each of the dozen people can be inferred based on their arousal and valence analyzed from the facial expressions and physiological responses. Certain web page designs can be understood by the retailer to cause viewers to be more favorable to specific products and even to make a buying decision more quickly. Alternatively, web pages which cause confusion can be replaced with web pages which can cause viewers to respond with confidence.

An aggregating machine 270 can be part of the system 200. Other sources of data 272 can be provided as input to the system 200 and can be used to aid in the cognitive state evaluation for the individual on whom the data collection 210 was performed. The other data sources 272 can include newsfeeds, Facebook™ pages, Twitter™, Flickr™, and other social networking and media. The aggregating machine 270 can analyze these other data sources 272 to aid in the evaluation of the cognitive state of the individual on which the data was collected.

For example, an employee of a company opts in to a self-assessment program where his or her face and electrodermal activity are monitored while performing job duties. The employee can also opt in to a tool where the aggregator 270 reads blog posts and social networking posts for mentions of the job, company, mood, or health. Over time, the employee is able to review social networking presence in context of perceived feelings for that day at work. The employee can also see how his or her mood and attitude can affect what is posted. One embodiment could be non-invasive, such as just counting the number of social network posts, or invasive, such as pumping the social networking content through an analysis engine that infers cognitive state from textual content.

In another example, a company might want to understand how news stories about the company in the Wall Street Journal™ and other publications affect employee morale and job satisfaction. The aggregator 270 can be programmed to search for news stories mentioning the company and link them back to the employees participating in this experiment. A person doing additional analysis can view the news stories about the company to provide additional context to each participant's cognitive state.

In yet another example, a facial analysis tool can process facial action units and gestures to infer cognitive states. As images are stored, metadata can be attached, such as the name of the person whose face is in a video that is part of the facial analysis. This video and metadata can be passed through a facial recognition engine which can be taught the face of the person. Once the face is recognizable to a facial recognition engine, the aggregator 270 can spider across the Internet, or just to specific websites such as Flickr™ and Facebook™, to find links with the same face. The additional pictures of the person located by facial recognition can be resubmitted to the facial analysis tool for an analysis to provide deeper insight into the subject's cognitive state.

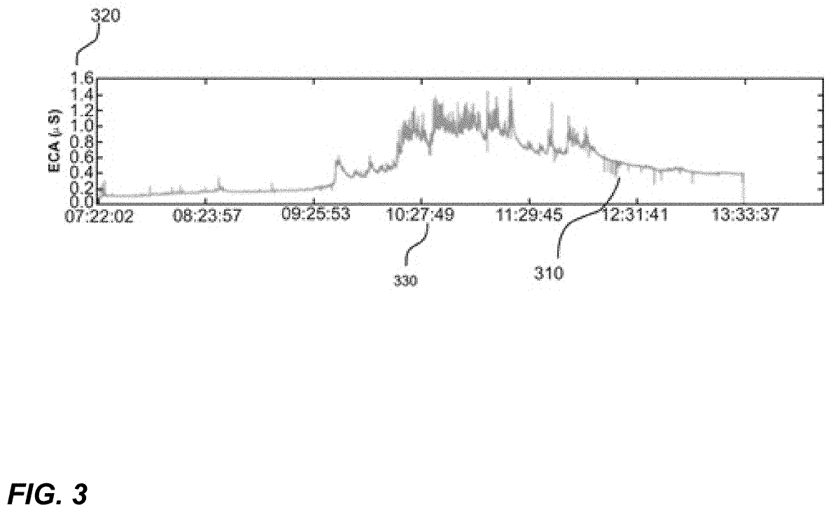

FIG. 3 is a graphical rendering of electrodermal activity. Electrodermal activity can include skin conductance which, in some embodiments, is measured in the units of micro-Siemens. A graph line 310 shows the electrodermal activity collected for an individual. The value for electrodermal activity is shown on the y-axis 320 for the graph. The electrodermal activity was collected over a period of time and the timescale 330 is shown on the x-axis of the graph. In some embodiments, electrodermal activity for multiple individuals is displayed when desired or shown on an aggregated basis. Markers can be included and can identify a section of the graph. The markers can be used to delineate a section of the graph that is or can be expanded. The expansion can cover a short period of time on which further analysis or review can be focused. This expanded portion can be rendered in another graph. Markers can also be included to identify sections corresponding to specific cognitive states. Each waveform or timeline can be annotated. A beginning annotation and an ending annotation can mark the beginning and end of a region or timeframe. A single annotation can mark a specific point in time. Each annotation can have associated text which was entered automatically or entered by a user. A text box can be displayed which includes the text.

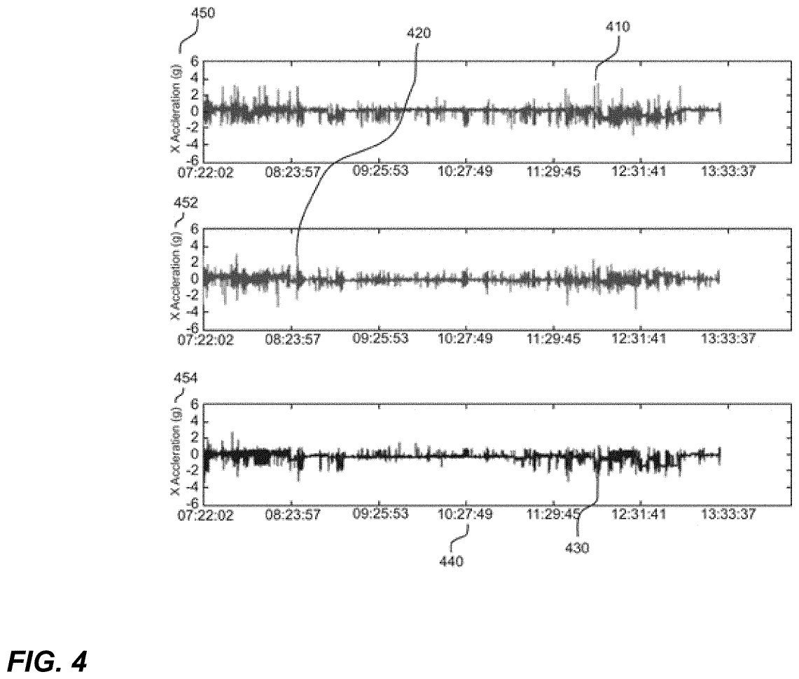

FIG. 4 is a graphical rendering of accelerometer data. One, two, or three dimensions of accelerometer data can be collected. In the example of FIG. 4, a graph for x-axis accelerometer readings is shown in a first graph 410, a graph for y-axis accelerometer readings is shown in a second graph 420, and a graph for z-axis accelerometer readings is shown in a third graph 430. The timestamps for the corresponding accelerometer readings are shown on a graph axis 440. The x acceleration values are shown on another axis 450 with the y acceleration values 452 and z acceleration values 454 shown as well. In some embodiments, accelerometer data for multiple individuals is displayed when desired or shown on an aggregated basis. Markers and annotations can be included and used similarly to those discussed in FIG. 3.

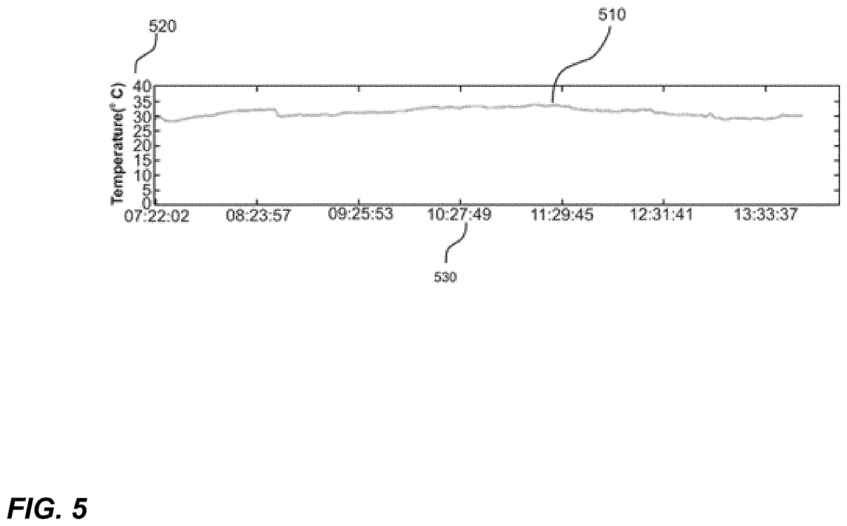

FIG. 5 is a graphical rendering of skin temperature data. A graph line 510 shows the electrodermal activity collected for an individual. The value for skin temperature is shown on the y-axis 520 for the graph. The skin temperature value was collected over a period of time and the timescale 530 is shown on the x-axis of the graph. In some embodiments, skin temperature values for multiple individuals are displayed when desired or shown on an aggregated basis. Markers and annotations can be included and used similarly to those discussed in FIG. 3.

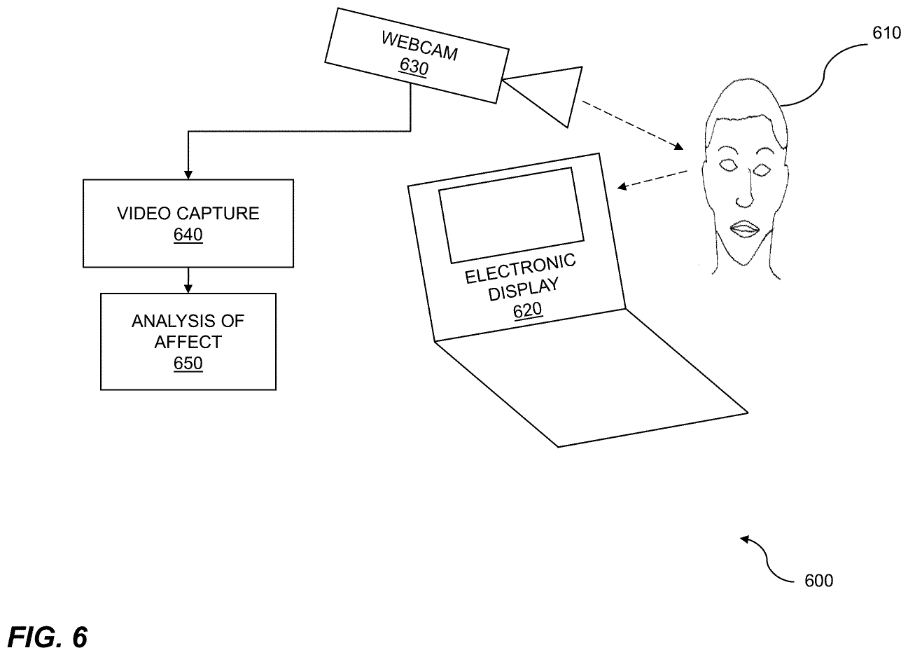

FIG. 6 shows an image collection system for facial analysis. The system 600 can enable distributed analysis for cognitive state metrics. The system 600 includes an electronic display 620 and a webcam 630. The system 600 captures a facial response to the electronic display 620. In some embodiments, the system 600 captures facial responses to other stimuli such as a store display, an automobile ride, a board game, a movie screen, or another experience. The facial data can include video and collection of information relating to cognitive states. In some embodiments, a webcam 630 captures video of the person 610. The video can be captured onto a disk, tape, into a computer system, or streamed to a server. Images or a sequence of images of the person 610 can be captured by a video camera, web camera still shots, a thermal imager, CCD devices, a phone camera, or another camera type apparatus.

The electronic display 620 can show a video or another presentation. The electronic display 620 can include a computer display, a laptop screen, a mobile device display, a cell phone display, or some other electronic display. The electronic display 620 can include a keyboard, mouse, joystick, touchpad, touch screen, wand, motion sensor, and other means of input. The electronic display 620 can show a webpage, a website, a web-enabled application, or the like. The images of the person 610 can be captured by a video capture unit 640. In some embodiments, video of the person 610 is captured, while in others, a series of still images is captured. In embodiments, a webcam is used to capture the facial data.

Analysis of action units, gestures, and cognitive states can be accomplished using the captured images of the person 610. The action units can be used to identify smiles, frowns, and other facial indicators of cognitive states. In some embodiments, smiles are directly identified, and in some cases the degree of smile (small, medium, and large, for example) can be identified. The gestures, including head gestures, can indicate interest or curiosity. For example, a head gesture of moving toward the electronic display 620 can indicate increased interest or a desire for clarification. Facial affect analysis 650 can be performed based on the information and images which are captured. The analysis can include facial analysis and analysis of head gestures. Based on the captured images, analysis of physiology can be performed. The evaluating of physiology can include evaluating heart rate, heart rate variability, respiration, perspiration, temperature, skin pore size, and other physiological characteristics by analyzing images of a person's face or body. In many cases, the evaluating can be accomplished using a webcam. Additionally, in some embodiments, physiology sensors are attached to the person to obtain further data on cognitive states.

The analysis can be performed in real time or “just in time”. In some embodiments, analysis is scheduled and then run through an analyzer or a computer processor which has been programmed to perform facial analysis. In some embodiments, the computer processor is aided by human intervention. The human intervention can identify cognitive states which the computer processor did not. In some embodiments, the processor identifies places where human intervention is useful, while in other embodiments, a person reviews the facial video and provides input even when the processor did not identify that intervention was useful. In some embodiments, the processor performs machine learning based on the human intervention. Based on the human input, the processor can learn that certain facial action units or gestures correspond to specific cognitive states and then can identify these cognitive states in an automated fashion without human intervention in the future.

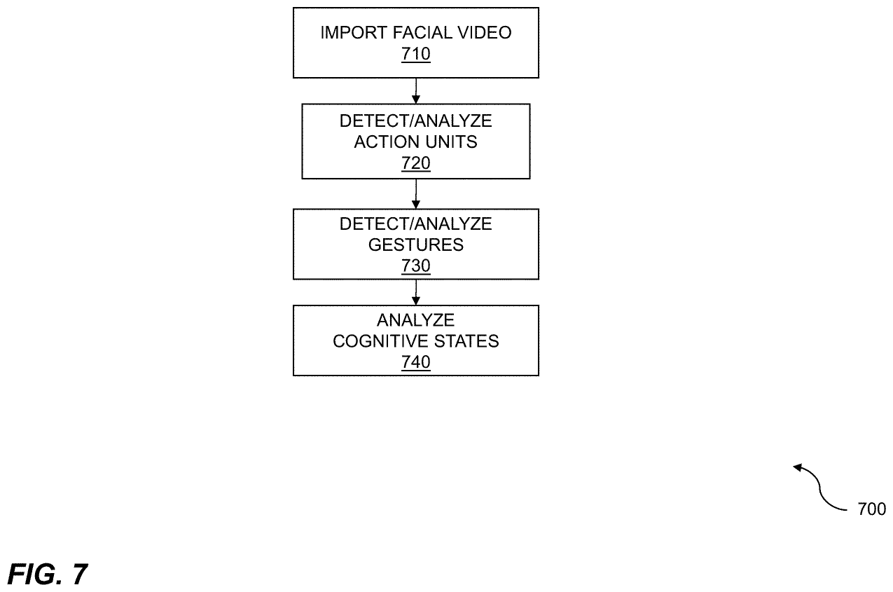

FIG. 7 is a flow diagram for performing facial analysis. The flow 700 can enable distributed analysis for cognitive state metrics. The flow 700 begins with importing of facial video 710. The facial video can have been previously recorded and stored for later analysis. Alternatively, the importing of facial video can occur in real time as an individual is being observed. The flow 700 continues with action units being detected and analyzed 720. Action units can include the raising of an inner eyebrow, tightening of the lip, lowering of the brow, flaring of nostrils, squinting of the eyes, and many other possibilities. These action units can be automatically detected by a computer system that is analyzing the video. Alternatively, small regions of motion of the face that are not traditionally numbered on formal lists of action units can also be considered as action units for input to the analysis, such as a twitch of a smile or an upward movement above both eyes. Furthermore, a combination of automatic detection by a computer system and human input can be provided to enhance the detection of the action units or related input measures. The flow 700 continues with facial and head gestures being detected and analyzed 730. Gestures can include tilting the head to the side, leaning forward, smiling, frowning, as well as many other gestures. In the flow 100, an analysis of cognitive states 740 is performed. The cognitive states can include happiness, sadness, concentration, confusion, as well as many others. Based on the action units and facial or head gestures, cognitive states can be analyzed, inferred, and identified.

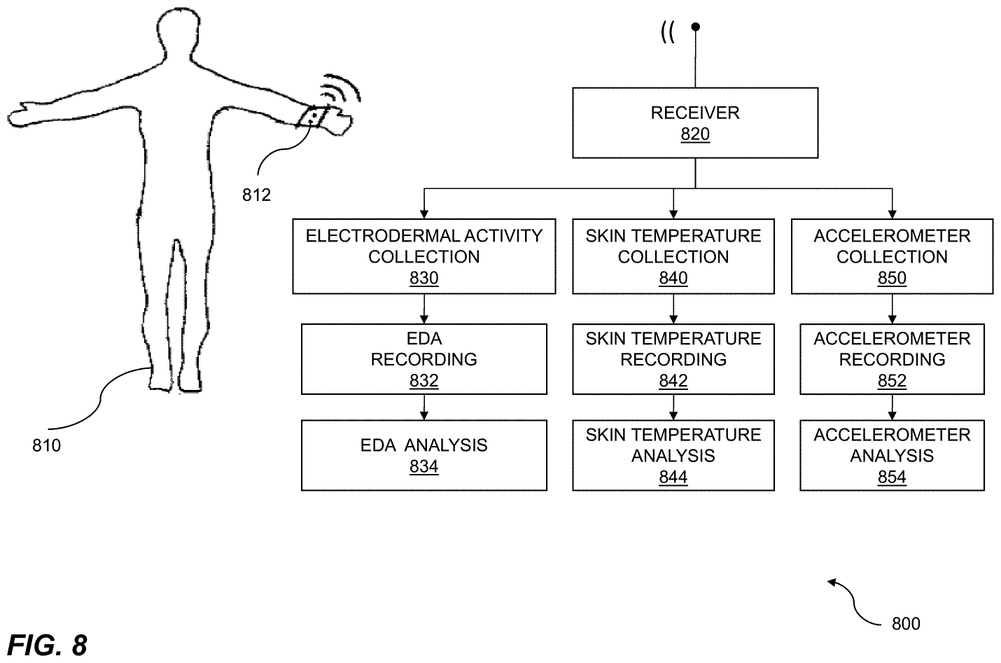

FIG. 8 is a diagram describing physiological analysis. A system 800 can analyze a person 810 for whom data is being collected. The person 810 can have a sensor 812 attached to him or her. The sensor 812 can be placed on the wrist, palm, hand, head, sternum, or another part of the body. In some embodiments, multiple sensors are placed on a person, such as on both wrists. The sensor 812 can include detectors for electrodermal activity, skin temperature, and accelerometer readings. Other detectors can also be included, such as heart rate, blood pressure, and other physiological detectors. The sensor 812 can transmit collected information to a receiver 820 using wireless technology such as Wi-Fi, Bluetooth, 802.11, cellular, or other bands. In some embodiments, the sensor 812 stores information and burst downloads the data through wireless technology. In other embodiments, the sensor 812 stores information for a later wired download. The receiver can provide the data to one or more components in the system 800. Electrodermal activity (EDA) can be collected 830. Electrodermal activity can be collected continuously, every second, four times per second, eight times per second, 32 times per second, on some other periodic basis, or based on some event. The electrodermal activity can be recorded 832. The recording can be recorded to a disk, to a tape, onto a flash drive, or into a computer system, or can be streamed to a server. The electrodermal activity can be analyzed 834. The electrodermal activity can indicate arousal, excitement, boredom, or other cognitive states based on changes in skin conductance.

Skin temperature can be collected 840 continuously, every second, four times per second, eight times per second, 32 times per second, or on some other periodic basis. The skin temperature can be recorded 842. The recording can be recorded to a disk, to a tape, onto a flash drive, or into a computer system, or can be streamed to a server. The skin temperature can be analyzed 844. The skin temperature can be used to indicate arousal, excitement, boredom, or other cognitive states based on changes in skin temperature.

Accelerometer data can be collected 850. The accelerometer can indicate one, two, or three dimensions of motion. The accelerometer data can be recorded 852. The recording can be recorded to a disk, to a tape, onto a flash drive, into a computer system, or can be streamed to a server. The accelerometer data can be analyzed 854. The accelerometer data can be used to indicate a sleep pattern, a state of high activity, a state of lethargy, or another state based on accelerometer data.

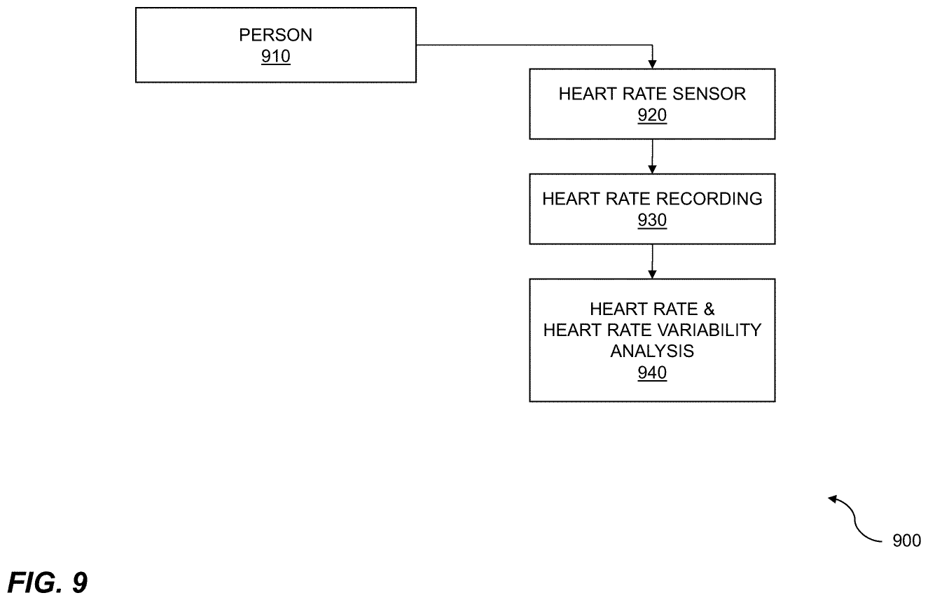

FIG. 9 is a flow diagram describing heart rate analysis. The flow 900 includes observing a person 910. The person can be observed by a heart rate sensor 920. The observation can be implemented through a contact sensor, through video analysis which enables capture of heart rate information, or through another method contactless sensing. The heart rate can be recorded 930. The recording can be recorded to a disk, to a tape, onto a flash drive, or into a computer system, or can be streamed to a server. The heart rate and heart rate variability can be analyzed 940. An elevated heart rate can indicate excitement, nervousness, or other cognitive states. A lowered heart rate can be used to indicate calmness, boredom, or other cognitive states. A heart rate being variable can indicate good health and lack of stress. A lack of heart rate variability can indicate an elevated level of stress.

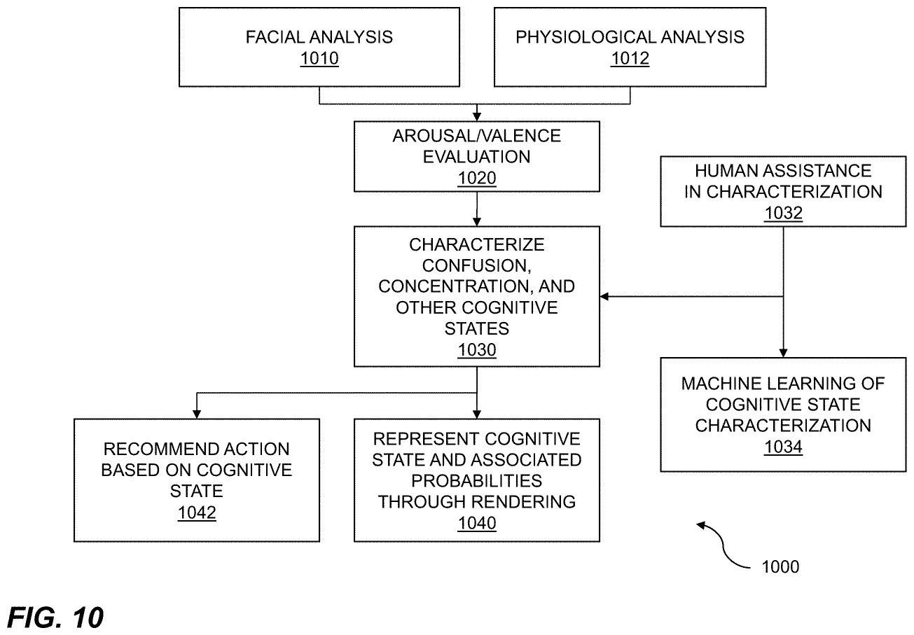

FIG. 10 is a flow diagram for performing cognitive state analysis and rendering. The flow 1000 can be used to enable distributed analysis of cognitive state metrics. The flow 1000 can begin with various types of data collection and analysis. Facial analysis 1010 can be performed, identifying action units, facial and head gestures, smiles, and cognitive states. Physiological analysis 1012 can be performed. The physiological analysis can include electrodermal activity, skin temperature, accelerometer data, heart rate, and other measurements related to the human body. The physiological data can be collected through contact sensors; through video analysis, as in the case of heart rate information; or through another means. In some embodiments, an arousal and valence evaluation 1020 is performed. A level of arousal can range from being calm to being excited. A valence can be a positive or a negative predisposition. The combination of valence and arousal can be used to characterize cognitive states 1030, and the cognitive states can include confusion, concentration, happiness, contentedness, confidence, as well as other states.

In some embodiments, the characterization of cognitive states 1030 is completely evaluated by a computer system. In other embodiments, human assistance is provided in inferring the cognitive state 1032. The process can involve using a human to evaluate a portion of facial expressions, head gestures, hand gestures, or body language. A human can be used to evaluate only a small portion or even a single expression or gesture. Thus, a human can evaluate a small portion of the facial expressions, head gestures, or hand gestures. Likewise, a human can evaluate a portion of the body language of the person being observed. In embodiments, the process involves prompting a person for input on an evaluation of the cognitive state for a section of the data which was captured. A person can view the facial analysis or physiological analysis raw data, including video, or can view portions of the raw data or analyzed results. The person can intervene and provide input to aid in the inferring of the cognitive state or can identify the cognitive state to the computer system used in the characterization of the cognitive state 1030. A computer system can highlight the portions of data where human intervention is needed and can jump to the point in time where the data for that needed intervention can be presented to the human. A feedback can be provided to the person that provides assistance in characterization. Multiple people can provide assistance in characterizing cognitive states. Based on the automated characterization of cognitive states as well as evaluation by multiple people, feedback can be provided to a person to improve her or his accuracy in characterization. Individuals can be compensated for providing assistance in characterization. Improved accuracy in characterization, based on the automated characterization or based on the other people assisting in characterization, can result in enhanced compensation.

The flow 1000 can include learning by the computer system. Machine learning of the cognitive state evaluation 1034 can be performed by the computer system used in the characterization of the cognitive state 1030. The machine learning can be based on the input from the person and on the evaluation of the cognitive state for the section of data.

A representation of the cognitive state and associated probabilities can be rendered 1040. The cognitive state can be presented on a computer display, electronic display, cell phone display, personal digital assistance screen, or another display. The cognitive state can be displayed graphically. A series of cognitive states can be presented with the likelihood of each state for a given point in time. Likewise, a series of probabilities for each cognitive state can be presented over the timeline for which facial and physiological data was analyzed. In some embodiments, an action is recommended based on the cognitive state 1042 which was detected. An action can include recommending a question in a focus group session, changing an advertisement on a web page, editing a movie which was viewed to remove an objectionable section or boring portion, moving a display in a store, or editing a confusing section of a tutorial on the web or in a video.

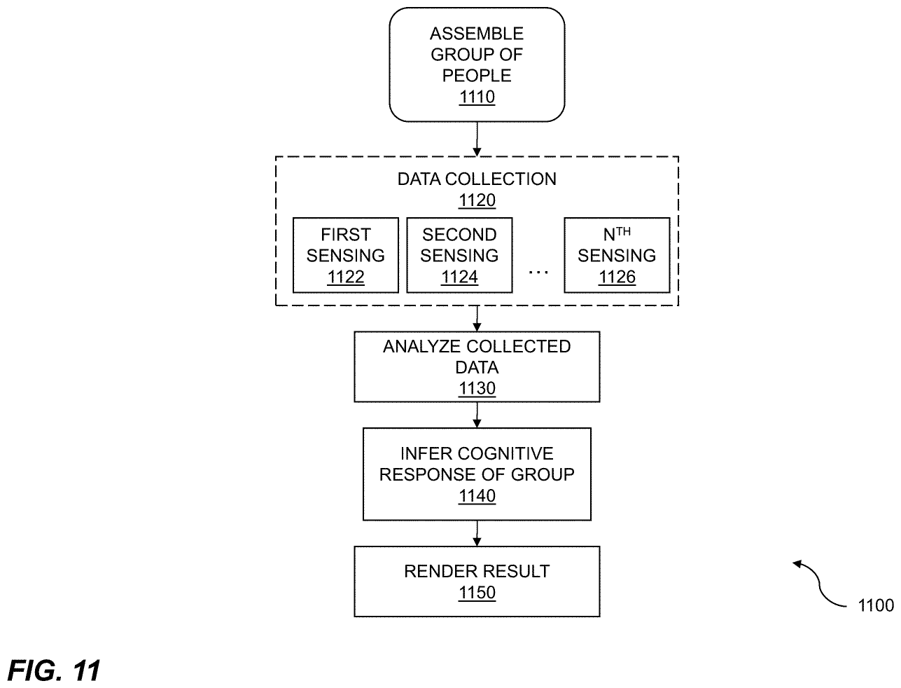

FIG. 11 is a flow diagram describing analysis of the mental response of a group. The flow 1100 can be used to enable distributed analysis for cognitive state metrics. The flow 1100 can begin with assembling a group of people 1110. The group of people can have a common experience such as viewing a movie, viewing a television show, viewing a movie trailer, viewing a streaming video, viewing an advertisement, listening to a song, viewing or listening to a lecture, using a computer program, using a product, consuming a food, using a video or computer game, participating in an educational experience through distance learning, riding in or driving a transportation vehicle such as a car, or some other experience. Data collection 1120 can be performed on each member of the group of people 1110. A plurality of sensings can occur on each member of the group of people 1110 including, for example, a first sensing 1122, a second sensing 1124, and so on through an n^{th} sensing 1126. The various sensings for which data collection 1120 is performed can include capturing facial expressions, electrodermal activity, skin temperature, accelerometer readings, heart rate, as well as other physiological information. The data which was captured can be analyzed 1130. This analysis can include characterization of arousal and valence as well as characterization of cognitive states for each of the individuals in the group of people 1110. The mental response of the group can be inferred 1140 providing a collective cognitive state. The cognitive states can be summarized to evaluate the common experience of all the individuals in the group of people 1110. A result can be rendered 1150. The result can be a function of time or a function of the sequence of events experienced by the people. The result can include a graphical display of the valence and arousal. The result can include a graphical display of the cognitive states of the individuals and the group collectively.

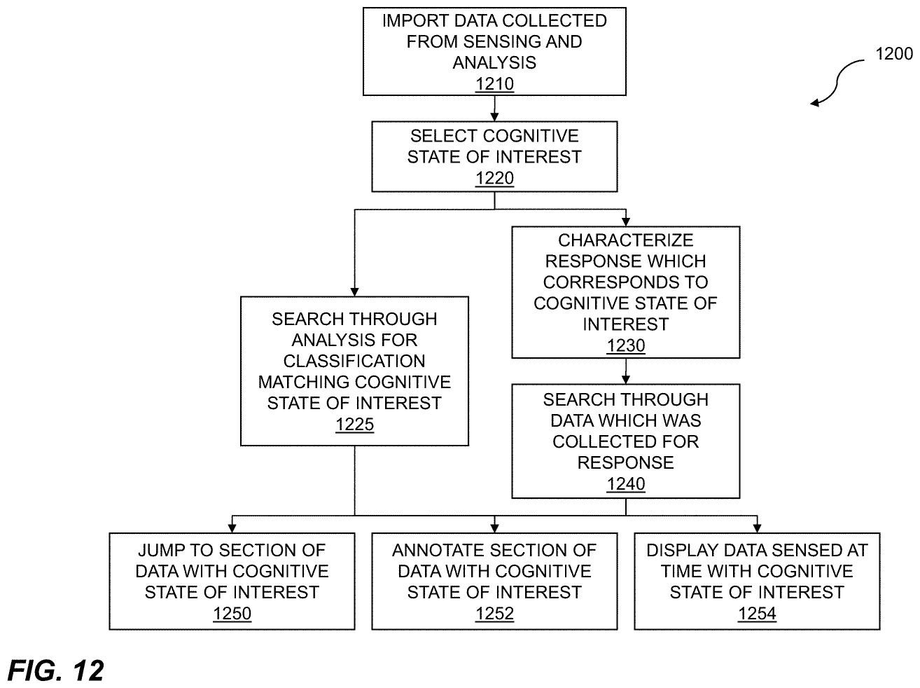

FIG. 12 is a flow diagram for identifying data portions which match a selected cognitive state of interest. The flow 1200 can be used in support of distributed analysis of cognitive state metrics. The flow 1200 begins with an import of data collected from sensing along with any analysis performed to date 1210. The importing of data can be the loading of stored data which was previously captured or can be the loading of data which is captured in real time. The data can also already exist within the system doing the analysis. The sensing can include capture of facial expressions, electrodermal activity, skin temperature, accelerometer readings, heart rate capture, as well as other physiological information. Analysis can be performed on the various data collected, from sensing to characterizing cognitive states.

A cognitive state that interests the user can be selected 1220. The cognitive state of interest can be confusion, concentration, confidence, delight, as well as many others. In some embodiments, analysis was previously performed on the data which was collected. The analysis can include indexing of the data and classifying cognitive states which were inferred or detected. When analysis has been previously performed and the cognitive state of interest has already been classified, a search through the analysis for one or more classifications matching the selected state can be performed 1225. By way of example, confusion can have been selected as the cognitive state of interest. The data which was collected can have been previously analyzed for various cognitive states, including confusion. When the data which was collected was indexed, a classification for confusion can have been tagged at various points in time during the data collection. The analysis can then be searched for any confusion points, as they have already been classified previously.

In some embodiments, a response is characterized which corresponds to the cognitive state of interest 1230. The response can be a positive valence and being aroused, as in an example where confidence is selected as the cognitive state of interest. The response can be reduced to valence and arousal or can be reduced further to look for action units or facial expressions and head gestures.

The data which was collected can be searched through for a response 1240 corresponding to the selected state. The sensed data can be searched, or derived analysis from the collected data can be searched. The search can look for action units, facial expressions, head gestures, or cognitive states which match the selected state for which the user is interested 1220.

The section of data with the cognitive state of interest can be jumped to 1250. For example, when confusion is selected, the data or analysis derived from the data can be shown corresponding to the point in time where confusion was exhibited. This “jump to feature” can be thought of as a fast-forward through the data to the interesting section where confusion or another selected cognitive state is detected. When facial video is considered, the key sections of the video which match the selected state can be displayed. In some embodiments, the section of the data with the cognitive state of interest is annotated 1252. Annotations can be placed along the timeline marking the data and the times with the selected state. In embodiments, the data sensed at the time with the selected state is displayed 1254. The data can include facial video. The data can also include graphical representation of electrodermal activity, skin temperature, accelerometer readouts, heart rate, and other physiological readings.

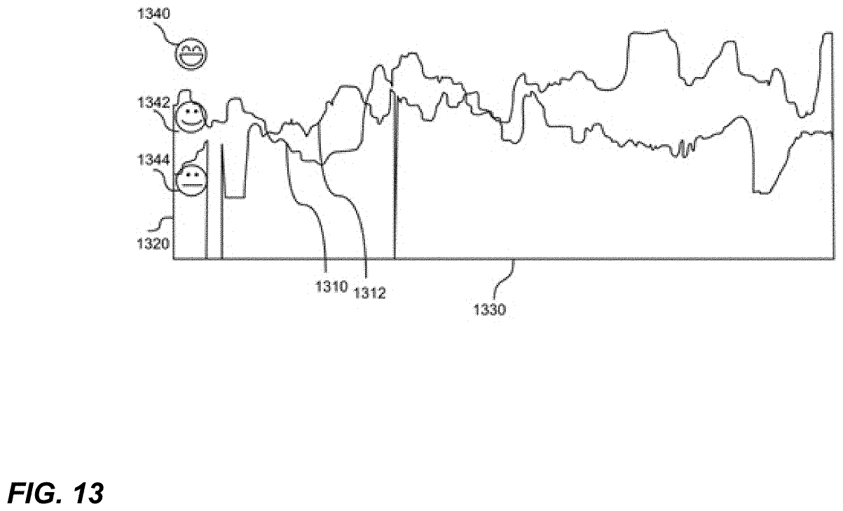

FIG. 13 is a graphical rendering of cognitive state analysis along with an aggregated result from a group of people. This rendering can be displayed on a web page, a web-enabled application, or another type of electronic display representation. A graph 1310 can be shown for an individual on whom affect data is collected. The cognitive state analysis can be based on facial image or physiological data collection. In some embodiments, the graph 1310 indicates the amount or probability of a smile being observed for the individual. A higher value or point on the graph can indicate a stronger or larger smile. In certain spots, the graph can drop out or degrade when image collection was lost or was not able to identify the face of the person. The probability or intensity of an affect can be given along the y-axis 1320. A timeline can be given along the x-axis 1330. Another graph 1312 can be shown for affect collected on another individual or for aggregated affect from multiple people. The aggregated information can be based on taking the average, median, or another collected value from a group of people. In some embodiments, graphical smiley face icons 1340, 1342, and 1344 are shown, providing an indication of the amount of a smile or another facial expression. A first broad smiley face icon 1340 can indicate a very large smile being observed. A second normal smiley face icon 1342 can indicate a smile being observed. A third face icon 1340 can indicate no smile. Each of the icons can correspond to a region on the y-axis 1320 that indicates the probability or intensity of a smile.

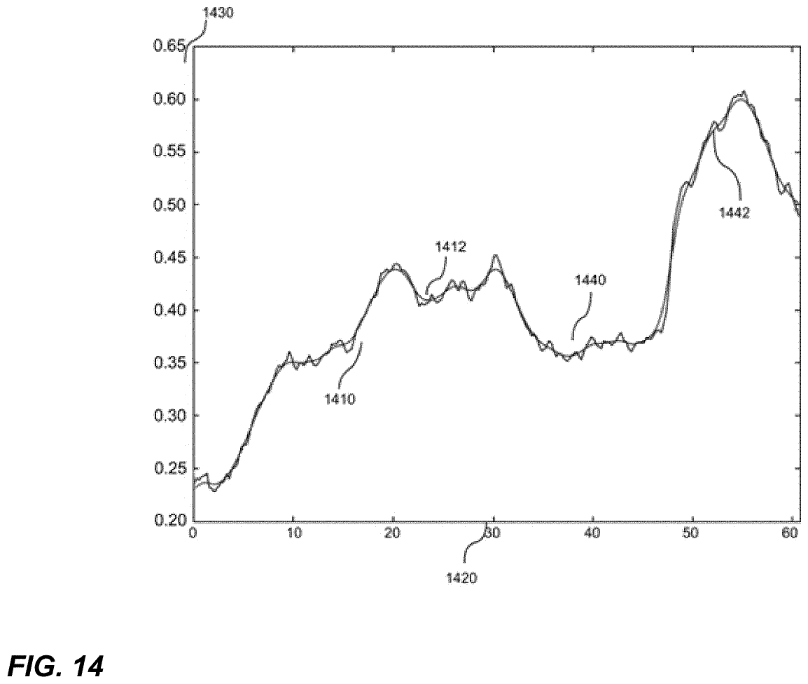

FIG. 14 is a graphical rendering of cognitive state analysis. This rendering can be displayed on a web page, a web-enabled application, or another type of electronic display representation. A graph 1410 can indicate the observed affect intensity or probability of occurring. A timeline can be given along the x-axis 1420. The probability or intensity of an affect can be given along the y-axis 1430. A second graph 1412 can show a smoothed version of the graph 1410. One or more valleys in the affect can be identified, such as the valley 1440. One or more peaks in affect can be identified, such as the peak 1442.

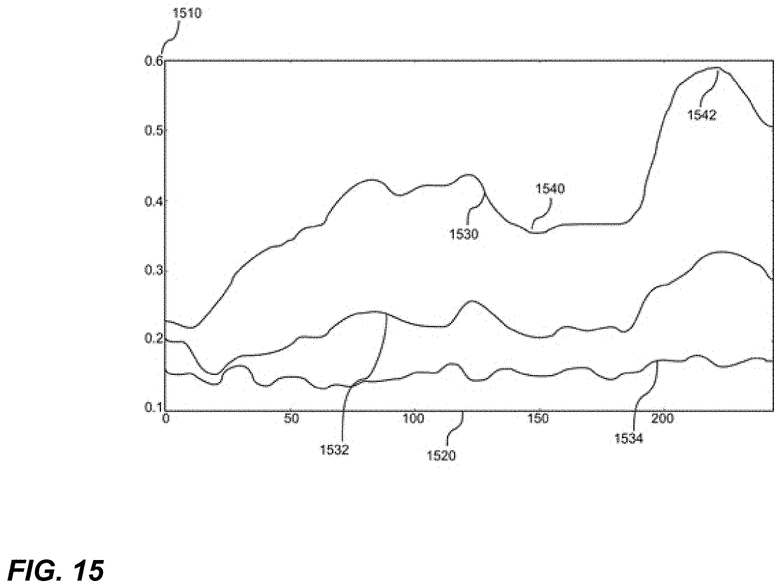

FIG. 15 is a graphical rendering of cognitive state analysis based on metadata. This rendering can be displayed on a web page, a web-enabled application, or another type of electronic display representation. On a graph 1510, a first line 1530, a second line 1532, and a third line 1534 can each correspond to different metadata collected. For instance, self-reporting metadata can be collected for whether the person reported that they “really liked”, “liked”, or “was ambivalent” about a certain event. The event could be a movie, a television show, a web series, a webisode, a video, a video clip, an electronic game, an advertisement, an e-book, an e-magazine, or the like. The first line 1530 can correspond to an event a person “really liked”, while the second line 1532 can correspond to another person who “liked” the event. Likewise, the third line 1534 can correspond to a different person who “was ambivalent” to the event. In some embodiments, the lines correspond to aggregated results of multiple people. One or more valleys in the affect can be identified, such as the valley 1540. One or more peaks in affect can also be identified, such as the peak 1542.

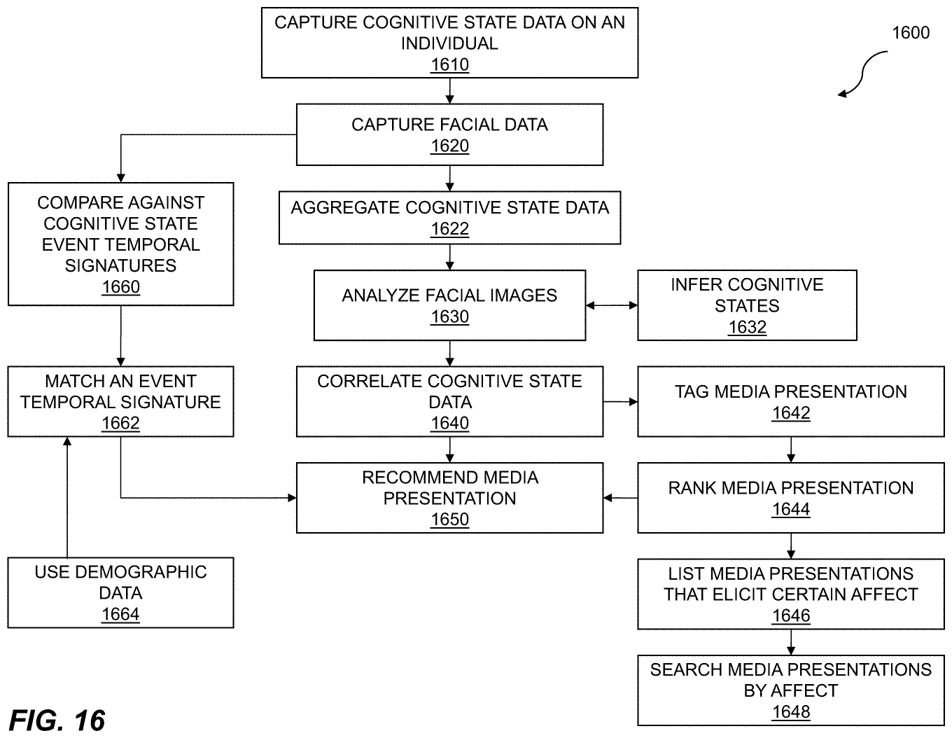

FIG. 16 is a flow diagram for cognitive state-based recommendations. The flow 1600 describes a computer-implemented method for affect-based, or cognitive state-based, ranking that can be used in support of distributed analysis for cognitive state metrics. The flow 1600 begins with capturing cognitive state data on an individual 1610. The capturing can be based on displaying a plurality of media presentations to a group of people of which the individual is a part. The displaying can be done all at once or through multiple occurrences. The plurality of media presentations can include videos. The plurality of videos can include YouTube™ videos, Vimeo™ videos, or Netflix™ videos. Further, the plurality of media presentations can include a movie, a movie trailer, a television show, a web series, a webisode, a video, a video clip, an advertisement, a music video, an electronic game, an e-book, or an e-magazine. The flow 1600 continues with capturing facial data 1620. The facial data can identify a first face. The captured facial data can be from the individual or from the group of people of which the individual is a part while the plurality of media presentations is displayed. Thus, cognitive state data can be captured from multiple people. The affect data can include facial images. In some embodiments, the playing of the media presentations is done on a mobile device and the recording of the facial images is done with the mobile device. The flow 1600 includes aggregating the cognitive state data 1622 from the multiple people. The flow 1600 further includes analyzing the facial images 1630 for a facial expression. The facial expression can include a smile or a brow furrow. The flow 1600 can further comprise using the facial images to infer cognitive states 1632. The cognitive states can include frustration, confusion, disappointment, hesitation, cognitive overload, focusing, being engaged, attending, boredom, exploration, confidence, trust, delight, valence, skepticism, satisfaction, and the like.

The flow 1600 includes correlating the cognitive state data 1640 captured from the group of people who have viewed the plurality of media presentations and had their cognitive state data captured. The plurality of videos viewed by the group of people can have some common videos seen by each of the people in the group of people. In some embodiments, the plurality of videos does not include an identical set of videos. The flow 1600 can continue with tagging the plurality of media presentations 1642 with cognitive state information based on the cognitive state data which was captured. In some embodiments, the affect information is simply the affect data, while in other embodiments, the affect information is the inferred cognitive states. In still other embodiments, the affect information is the results of the correlation. The flow 1600 continues with ranking the media presentations 1644 relative to another media presentation based on the cognitive state data which was collected. The ranking can be for an individual based on the cognitive state data captured from the individual. The ranking can be based on anticipated preferences for the individual. In some embodiments, the ranking of a first media presentation relative to another media presentation is based on the cognitive state data which was aggregated from multiple people. The ranking can also be relative to media presentations previously stored with affect information. The ranking can include ranking a video relative to another video based on the cognitive state data which was captured. The flow 1600 can further include displaying the videos that elicit a certain affect 1646. The certain affect can include smiles, engagement, attention, interest, sadness, liking, disliking, and so on. The ranking can further comprise displaying the videos which elicited a larger number of smiles. Because of ranking, the media presentations can be sorted based on which videos are the funniest; which videos are the saddest and generate the most tears; or which videos which engender some other response. The flow 1600 can further include searching through the videos based on a certain affect data 1648. A search can identify videos which are very engaging, funny, sad, poignant, or the like.

The flow 1600 includes comparing the cognitive state data that was captured for the individual against a plurality of cognitive state event temporal signatures 1660. In embodiments, multiple cognitive state event temporal signatures have been obtained from previous analysis of numerous people. The cognitive state event temporal signatures can include information on rise time to facial expression intensity, fall time from facial expression intensity, duration of a facial expression, and so on. In some embodiments, the cognitive state event temporal signatures are associated with certain demographics, ethnicities, cultures, etc. The cognitive state event temporal signatures can be used to identify one or more of sadness, stress, happiness, anger, frustration, confusion, disappointment, hesitation, cognitive overload, focusing, engagement, attention, boredom, exploration, confidence, trust, delight, disgust, skepticism, doubt, satisfaction, excitement, laughter, calmness, curiosity, humor, depression, envy, sympathy, embarrassment, poignancy, or mirth. The cognitive state event temporal signatures can be used to identify liking or satisfaction with a media presentation. The cognitive state event temporal signatures can be used to correlate with appreciating a second media presentation. The flow 1600 can include matching a first event signature 1662, from the plurality of cognitive state event temporal signatures, against the cognitive state data that was captured. In embodiments, an output rendering is based on the matching of the first event signature. The matching can include identifying similar aspects of the cognitive state event temporal signature such as rise time, fall time, duration, and so on. The matching can include matching a series of facial expressions described in cognitive state event temporal signatures. In some embodiments, a second cognitive state event temporal signature is used to identify a sequence of cognitive state data being expressed by an individual. In some embodiments, demographic data 1664 is used to provide a demographic basis for analyzing temporal signatures. In some embodiments, the analysis includes demographic information distilled from the data.

The flow 1600 includes recommending a second media presentation 1650 to an individual based on the affect data that was captured and based on the ranking. The recommending the second media presentation to the individual is further based on the comparing of the cognitive state data to the plurality of cognitive state event temporal signatures. The second media presentation can be a movie, a movie trailer, a television show, a web series, a webisode, a video, a video clip, an advertisement, a music video, an electronic game, an e-book, or an e-magazine. The recommending the second media presentation can be further based on the matching of the first event signature. The recommending can be based on similarity of cognitive states expressed. The recommending can be based on a numerically quantifiable determination of satisfaction or appreciation of the first media and an anticipated numerically quantifiable satisfaction or appreciation of second first media presentation.

Based on the cognitive states, recommendations to or from an individual can be provided. One or more recommendations can be made to the individual based on cognitive states, affect, or facial expressions. A correlation can be made between one individual and others with similar affect exhibited during multiple videos. The correlation can include a record of other videos, games, or other experiences, along with their affect. Likewise, a recommendation for a movie, video, video clip, webisode or another activity can be made to an individual based on their affect. Various steps in the flow 1600 may be changed in order, repeated, omitted, or the like without departing from the disclosed inventive concepts. Various embodiments of the flow 1600 may be included in a computer program product embodied in a non-transitory computer readable medium that includes code executable by one or more processors.

The human face provides a powerful communications medium through its ability to exhibit a myriad of expressions that can be captured and analyzed for a variety of purposes. In some cases, media producers are acutely interested in evaluating the effectiveness of message delivery by video media. Such video media includes advertisements, political messages, educational materials, television programs, movies, government service announcements, etc. Automated facial analysis can be performed on one or more video frames containing a face in order to detect facial action. Based on the detected facial action, a variety of parameters can be determined, including affect valence, spontaneous reactions, facial action units, and so on. The parameters that are determined can be used to infer or predict emotional and cognitive states. For example, determined valence can be used to describe the emotional reaction of a viewer to a video media presentation or another type of presentation. Positive valence provides evidence that a viewer is experiencing a favorable emotional response to the video media presentation, while negative valence provides evidence that a viewer is experiencing an unfavorable emotional response to the video media presentation. Other facial data analysis can include the determination of discrete emotional states of the viewer or viewers.

Facial data can be collected from a plurality of people using any of a variety of cameras. A camera can include a webcam, a video camera, a still camera, a thermal imager, a CCD device, a phone camera, a three-dimensional camera, a depth camera, a light field camera, multiple webcams used to show different views of a person, or any other type of image capture apparatus that can allow captured data to be used in an electronic system. In some embodiments, the person is permitted to “opt in” to the facial data collection. For example, the person can agree to the capture of facial data using a personal device such as a mobile device or another electronic device by selecting an opt-in choice. Opting-in can then turn on the person's webcam-enabled device and can begin the capture of the person's facial data via a video feed from the webcam or other camera. The video data that is collected can include one or more persons experiencing an event. The one or more persons can be sharing a personal electronic device or can each be using one or more devices for video capture. The videos that are collected can be collected using a web-based framework. The web-based framework can be used to display the video media presentation or event as well as to collect videos from any number of viewers who are online. That is, the collection of videos can be crowdsourced from those viewers who elected to opt in to the video data collection.

In some embodiments, a high frame rate camera is used. A high frame rate camera has a frame rate of sixty frames per second or higher. With such a frame rate, microexpressions can also be captured. Microexpressions are very brief facial expressions, lasting only a fraction of a second. They occur when a person either deliberately or unconsciously conceals a feeling.

In some cases, microexpressions occur when people have hidden their feelings from themselves (repression) or when they deliberately try to conceal their feelings from others. Sometimes the microexpressions might only last about fifty milliseconds. Hence, these expressions can go unnoticed by a human observer. However, a high frame-rate camera can be used to capture footage at a sufficient frame rate such that the footage can be analyzed for the presence of microexpressions. Microexpressions can be analyzed via action units as previously described, with various attributes such as brow raising, brow furrows, eyelid raising, and the like. Thus, embodiments analyze microexpressions that are easily missed by human observers due to their transient nature.

The videos captured from the various viewers who chose to opt in can be substantially different in terms of video quality, frame rate, etc. As a result, the facial video data can be scaled, rotated, and otherwise adjusted to improve consistency. Human factors further impact the capture of the facial video data. The facial data that is captured might or might not be relevant to the video media presentation being displayed. For example, the viewer might not be paying attention, might be fidgeting, might be distracted by an object or event near the viewer, or might be otherwise inattentive to the video media presentation. The behavior exhibited by the viewer can prove challenging to analyze due to viewer actions including eating, speaking to another person or persons, speaking on the phone, etc. The videos collected from the viewers might also include other artifacts that pose challenges during the analysis of the video data. The artifacts can include such items as eyeglasses (because of reflections), eye patches, jewelry, and clothing that occludes or obscures the viewer's face. Similarly, a viewer's hair or hair covering can present artifacts by obscuring the viewer's eyes and/or face.

The captured facial data can be analyzed using the facial action coding system (FACS). The FACS seeks to define groups or taxonomies of facial movements of the human face. The FACS encodes movements of individual muscles of the face, where the muscle movements often include slight, instantaneous changes in facial appearance. The FACS encoding is commonly performed by trained observers, but can also be performed on automated, computer-based systems. Analysis of the FACS encoding can be used to determine emotions of the persons whose facial data is captured in the videos. The FACS is used to encode a wide range of facial expressions that are anatomically possible for the human face. The FACS encodings include action units (AUs) and related temporal segments that are based on the captured facial expression. The AUs are open to higher order interpretation and decision-making. For example, the AUs can be used to recognize emotions experienced by the observed person. Emotion-related facial actions can be identified using the emotional facial action coding system (EMFACS) and the facial action coding system affect interpretation dictionary (FACSAID), for example. For a given emotion, specific action units can be related to the emotion. For example, the emotion of anger can be related to AUs 4, 5, 7, and 23, while happiness can be related to AUs 6 and 12. Other mappings of emotions to AUs have also been previously associated. The coding of the AUs can include an intensity scoring that ranges from A (trace) to E (maximum). The AUs can be used for analyzing images to identify patterns indicative of a particular mental and/or emotional state. The AUs range in number from 0 (neutral face) to 98 (fast up-down look). The AUs include so-called main codes (inner brow raiser, lid tightener, etc.), head movement codes (head turn left, head up, etc.), eye movement codes (eyes turned left, eyes up, etc.), visibility codes (eyes not visible, entire face not visible, etc.), and gross behavior codes (sniff, swallow, etc.). Emotion scoring can be included where intensity is evaluated in addition to specific emotions, moods, or cognitive states.

The coding of faces identified in videos captured of people observing an event can be automated. The automated systems can detect facial AUs or discrete emotional states. The emotional states can include amusement, fear, anger, disgust, surprise, and sadness, for example. The automated systems can be based on a probability estimate from one or more classifiers, where the probabilities can correlate with an intensity of an AU or an expression. The classifiers can be used to identify into which of a set of categories a given observation can be placed. For example, the classifiers can be used to determine a probability that a given AU or expression is present in a given frame of a video. The classifiers can be used as part of a supervised machine learning technique where the machine learning technique can be trained using “known good” data. Once trained, the machine learning technique can proceed to classify new data that is captured.

The supervised machine learning models can be based on support vector machines (SVMs). An SVM can have an associated learning model that is used for data analysis and pattern analysis. For example, an SVM can be used to classify data that can be obtained from collected videos of people experiencing a media presentation. An SVM can be trained using “known good” data that is labeled as belonging to one of two categories (e.g. smile and no-smile). The SVM can build a model that assigns new data into one of the two categories. The SVM can construct one or more hyperplanes that can be used for classification. The hyperplane that has the largest distance from the nearest training point can be determined to have the best separation. The largest separation can improve the classification technique by increasing the probability that a given data point can be properly classified.

In another example, a histogram of oriented gradients (HoG) can be computed. The HoG can include feature descriptors and can be computed for one or more facial regions of interest. The regions of interest of the face can be located using facial landmark points, where the facial landmark points can include outer edges of nostrils, outer edges of the mouth, outer edges of eyes, etc. A HoG for a given region of interest can count occurrences of gradient orientation within a given section of a frame from a video, for example. The gradients can be intensity gradients and can be used to describe an appearance and a shape of a local object. The HoG descriptors can be determined by dividing an image into small, connected regions, also called cells. A histogram of gradient directions or edge orientations can be computed for pixels in the cell. Histograms can be contrast-normalized based on intensity across a portion of the image or the entire image, thus reducing any influence from illumination or shadowing changes between and among video frames. The HoG can be computed on the image or on an adjusted version of the image, where the adjustment of the image can include scaling, rotation, etc. For example, the image can be adjusted by flipping the image around a vertical line through the middle of a face in the image. The symmetry plane of the image can be determined from the tracker points and landmarks of the image.

Embodiments include identifying a first face and a second face within the facial data. Identifying and analyzing can be accomplished without further interaction with the cloud environment, in coordination with the cloud environment, and so on. In an embodiment, an automated facial analysis system identifies five facial actions or action combinations in order to detect spontaneous facial expressions for media research purposes. Based on the facial expressions that are detected, a determination can be made with regard to the effectiveness of a given video media presentation, for example. The system can detect the presence of the AUs or the combination of AUs in videos collected from a plurality of people. The facial analysis technique can be trained using a web-based framework to crowdsource videos of people as they watch online video content. The video can be streamed at a fixed frame rate to a server. Human labelers can code for the presence or absence of facial actions including symmetric smile, unilateral smile, asymmetric smile, and so on. The trained system can then be used to automatically code the facial data collected from a plurality of viewers experiencing video presentations (e.g. television programs).

Spontaneous asymmetric smiles can be detected in order to understand viewer experiences. Related literature indicates that as many asymmetric smiles occur on the right hemi face as do on the left hemi face, for spontaneous expressions. Detection can be treated as a binary classification problem, where images that contain a right asymmetric expression are used as positive (target class) samples and all other images as negative (non-target class) samples. Classifiers perform the classification, including classifiers such as support vector machines (SVM) and random forests. Random forests can include ensemble-learning methods that use multiple learning algorithms to obtain better predictive performance. Frame-by-frame detection can be performed to recognize the presence of an asymmetric expression in each frame of a video. Facial points can be detected, including the top of the mouth and the two outer eye corners. The face can be extracted, cropped, and warped into a pixel image of specific dimension (e.g. 96×96 pixels). In embodiments, the inter-ocular distance and vertical scale in the pixel image are fixed. Feature extraction can be performed using computer vision software such as OpenCV™. Feature extraction can be based on the use of HoGs. HoGs can include feature descriptors and can be used to count occurrences of gradient orientation in localized portions or regions of the image. Other techniques can be used for counting occurrences of gradient orientation, including edge orientation histograms, scale-invariant feature transformation descriptors, etc. The AU recognition tasks can also be performed using Local Binary Patterns (LBPs) and Local Gabor Binary Patterns (LGBPs). The HoG descriptor represents the face as a distribution of intensity gradients and edge directions, and is robust in its ability to translate and scale. Differing patterns, including groupings of cells of various sizes and arranged in variously sized cell blocks, can be used. For example, 4×4 cell blocks of 8×8 pixel cells with an overlap of half of the block can be used. Histograms of channels can be used, including nine channels or bins evenly spread over 0-180 degrees. In this example, the HoG descriptor on a 96×96 image is 25 blocks×16 cells×9 bins=3600, the latter quantity representing the dimension. AU occurrences can be rendered. The videos can be grouped into demographic datasets based on nationality and/or other demographic parameters for further detailed analysis.

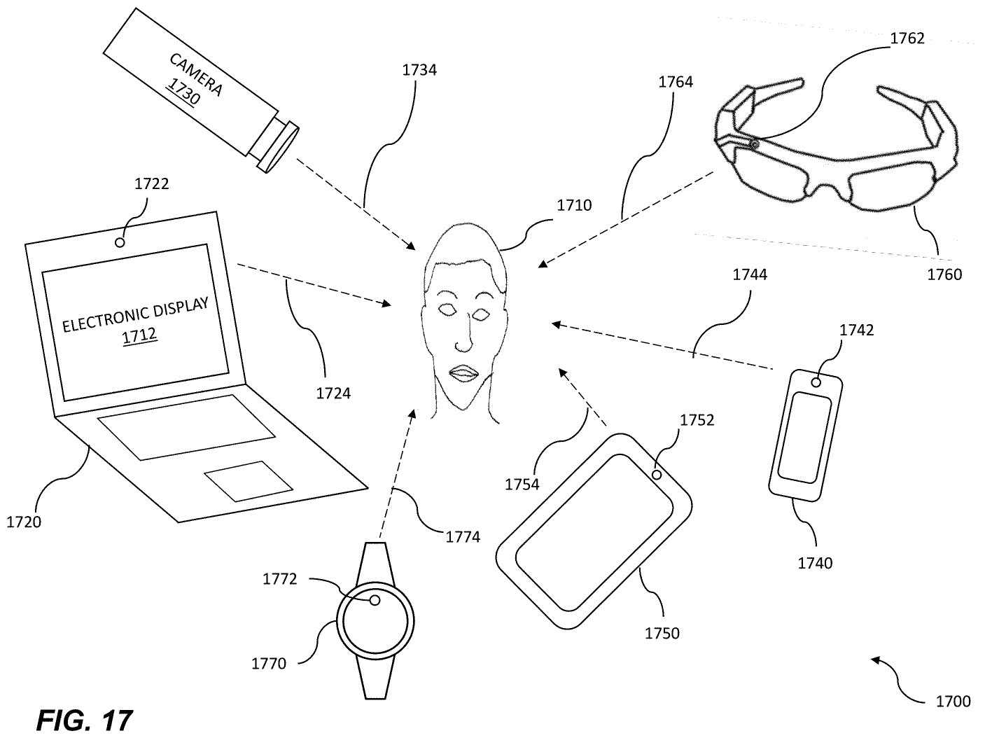

FIG. 17 shows example image collection including multiple mobile devices 1700. Image collection can be used in support of distributed analysis for cognitive state metrics. The images that can be collected can be analyzed to perform cognitive state analysis as well as to determine weights and image classifiers. The weights and the image classifiers can be used to infer an emotional metric. The multiple mobile devices can be used to collect video data on a person. While one person is shown, in practice, the video data can be collected on any number of people. A user 1710 can be observed as she or he is performing a task, experiencing an event, viewing a media presentation, and so on. The user 1710 can be viewing a media presentation or another form of displayed media. The one or more video presentations can be visible to a plurality of people instead of an individual user. If the plurality of people is viewing a media presentation, then the media presentations can be displayed on an electronic display 1712. The data collected on the user 1710 or on a plurality of users can be in the form of one or more videos. The plurality of videos can be of people who are experiencing different situations. Some example situations can include the user or plurality of users viewing one or more robots performing various tasks. The situations could also include exposure to media such as advertisements, political messages, news programs, and so on. As noted before, video data can be collected on one or more users in substantially identical or different situations. The data collected on the user 1710 can be analyzed and viewed for a variety of purposes, including expression analysis. The electronic display 1712 can be on a laptop computer 1720 as shown, a tablet computer 1750, a cell phone 1740, a television, a mobile monitor, or any other type of electronic device. In a certain embodiment, expression data is collected on a mobile device such as a cell phone 1740, a tablet computer 1750, a laptop computer 1720, or a watch 1770. Thus, the multiple sources can include at least one mobile device such as a cell phone 1740 or a tablet computer 1750, or a wearable device such as a watch 1770 or glasses 1760. A mobile device can include a forward-facing camera and/or a rear-facing camera that can be used to collect expression data. Sources of expression data can include a webcam 1722, a phone camera 1742, a tablet camera 1752, a wearable camera 1762, and a mobile camera 1730. A wearable camera can comprise various camera devices, such as the watch camera 1772.

As the user 1710 is monitored, the user 1710 might move due to the nature of the task, boredom, discomfort, distractions, or for another reason. As the user moves, the camera with a view of the user's face can change. Thus, as an example, if the user 1710 is looking in a first direction, the line of sight 1724 from the webcam 1722 is able to observe the individual's face, but if the user is looking in a second direction, the line of sight 1734 from the mobile camera 1730 is able to observe the individual's face. Further, in other embodiments, if the user is looking in a third direction, the line of sight 1744 from the phone camera 1742 is able to observe the individual's face, and if the user is looking in a fourth direction, the line of sight 1754 from the tablet camera 1752 is able to observe the individual's face. If the user is looking in a fifth direction, the line of sight 1764 from the wearable camera 1762, which can be a device such as the glasses 1760 shown and can be worn by another user or an observer, is able to observe the individual's face. If the user is looking in a sixth direction, the line of sight 1774 from the wearable watch-type device 1770 with a camera 1772 included on the device, is able to observe the individual's face. In other embodiments, the wearable device is another device, such as an earpiece with a camera, a helmet or hat with a camera, a clip-on camera attached to clothing, or any other type of wearable device with a camera or another sensor for collecting expression data. The user 1710 can also employ a wearable device including a camera for gathering contextual information and/or collecting expression data on other users. Because the user 1710 can move her or his head, the facial data can be collected intermittently when the individual is looking in a direction of a camera. In some cases, multiple people are included in the view from one or more cameras, and some embodiments include filtering out faces of one or more other people to determine whether the user 1710 is looking toward a camera. All or some of the expression data can be continuously or sporadically available from these various devices and other devices.

The captured video data can include facial expressions and can be analyzed on a computing device, such as the video capture device or on another separate device. The analysis of the video data can include the use of a classifier. For example, the video data can be captured using one of the mobile devices discussed above and sent to a server or another computing device for analysis. However, the captured video data including expressions can also be analyzed on the device which performed the capturing. For example, the analysis can be performed on a mobile device, where the videos were obtained with the mobile device and wherein the mobile device includes one or more of a laptop computer, a tablet, a PDA, a smartphone, a wearable device, and so on. In another embodiment, the analyzing comprises using a classifier on a server or other computing device other than the capturing device. The result of the analyzing can be used to infer one or more emotional metrics.

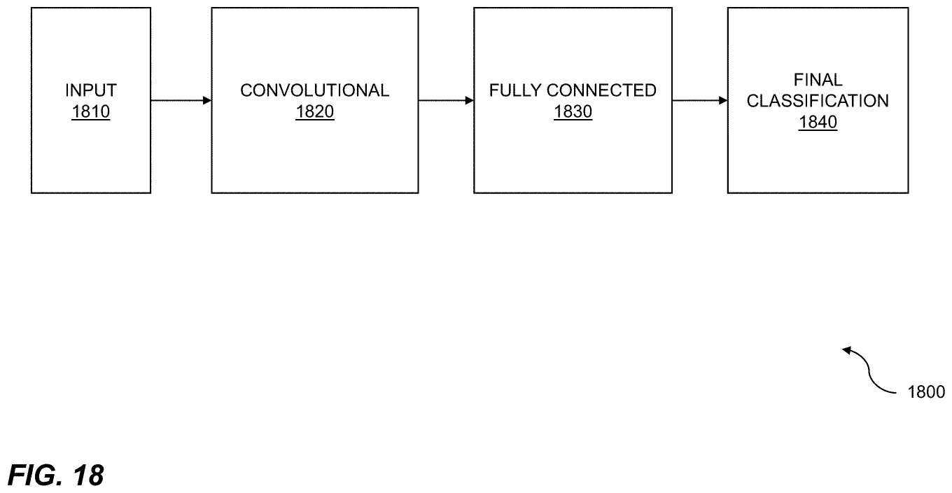

FIG. 18 is an example showing a pipeline for facial analysis layers. A pipeline of facial analysis layers can be applied to distributed analysis for cognitive state metrics. For example, a computer is initialized for convolutional neural network processing. A plurality of images for processing on the computer is obtained, using an imaging device. A multilayered analysis engine is trained on the computer, using the plurality of images. The multilayered analysis engine includes multiple layers that include one or more convolutional layers, one or more hidden layers, and at least one output layer. A further image is evaluated, using the multilayered analysis engine. The evaluating includes identifying a facial portion and identifying a facial expression based on the facial portion. The convolutional neural network analysis is output from the output layer. The example 1800 includes an input layer 1810. The input layer 1810 receives image data. The image data can be input in a variety of formats, such as JPEG, TIFF, BMP, and GIF. Compressed image formats can be decompressed into arrays of pixels, wherein each pixel can include an RGB tuple. The input layer 1810 can then perform processing such as identifying boundaries of the face, identifying landmarks of the face, extracting features of the face, and/or rotating a face within the plurality of images. The output of the input layer can then be input to a convolutional layer 1820. The convolutional layer 1820 can represent a convolutional neural network and can contain a plurality of hidden layers. A layer from the multiple layers can be fully connected. The convolutional layer 1820 can reduce the amount of data feeding into a fully connected layer 1830. The fully connected layer processes each pixel/data point from the convolutional layer 1820. A last layer within the multiple layers can provide output indicative of a certain cognitive state. The last layer is the final classification layer 1840. The output of the final classification layer 1840 can be indicative of the cognitive states of faces within the images that are provided to input layer 1810.

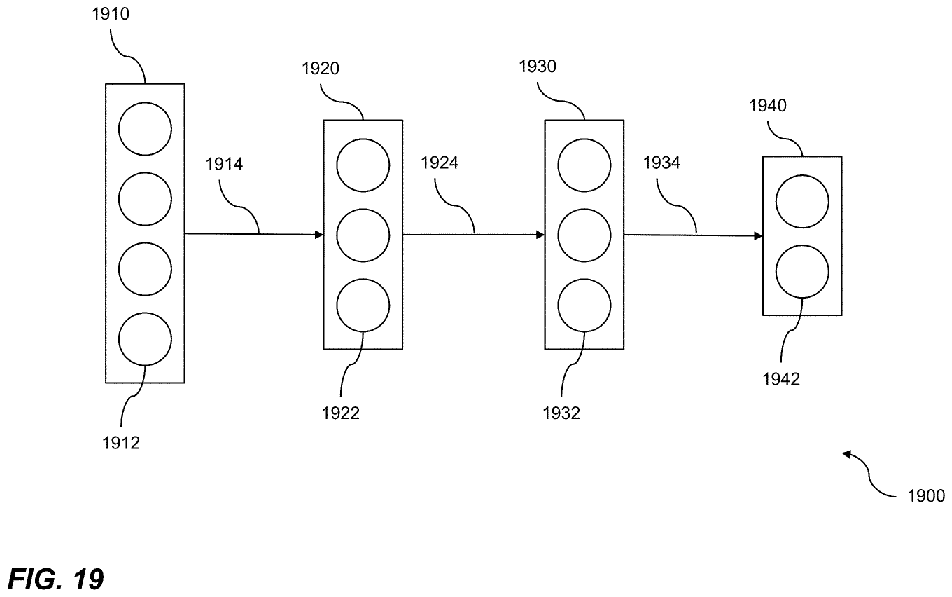

FIG. 19 is an example 1900 illustrating a deep network for facial expression parsing. Facial expression parsing using neural networks can be applied to distributed analysis for cognitive state metrics. Data for an individual is captured into a computing device. The data for the individual is uploaded to a web server. A cognitive state metric for the individual is calculated. Analysis from the web server is received by the computing device. The analysis is based on the data for the individual and the cognitive state metric for the individual. An output is rendered at the computing device that describes a cognitive state of the individual. A first layer 1910 of the deep network is comprised of a plurality of nodes 1912. Each of nodes 1912 serves as a neuron within a neural network. The first layer can receive data from an input layer. The output of the first layer 1910 feeds to the next layer 1920. The layer 1920 further comprises a plurality of nodes 1922. A weight 1914 adjusts the output of the first layer 1910 which is being input to the layer 1920. In embodiments, the layer 1920 is a hidden layer. The output of the layer 1920 feeds to a subsequent layer 1930. That layer 1930 further comprises a plurality of nodes 1932. A weight 1924 adjusts the output of the second layer 1920 which is being input to the third layer 1930. In embodiments, the third layer 1930 is also a hidden layer. The output of the third layer 1930 feeds to a fourth layer 1940 which further comprises a plurality of nodes 1942. A weight 1934 adjusts the output of the third layer 1930 which is being input to the fourth layer 1940. The fourth layer 1940 can be a final layer, providing a facial expression and/or cognitive state as its output. The facial expression can be identified using a hidden layer from the one or more hidden layers. The weights can be provided on inputs to the multiple layers to emphasize certain facial features within the face. The training can comprise assigning weights to inputs on one or more layers within the multilayered analysis engine. In embodiments, one or more of the weights (1914, 1924, and/or 1934) can be adjusted or updated during training. The assigning weights can be accomplished during a feed-forward pass through the multilayered analysis engine. In a feed-forward arrangement, the information moves forward from the input nodes through the hidden nodes and on to the output nodes. Additionally, the weights can be updated during a backpropagation process through the multilayered analysis engine.

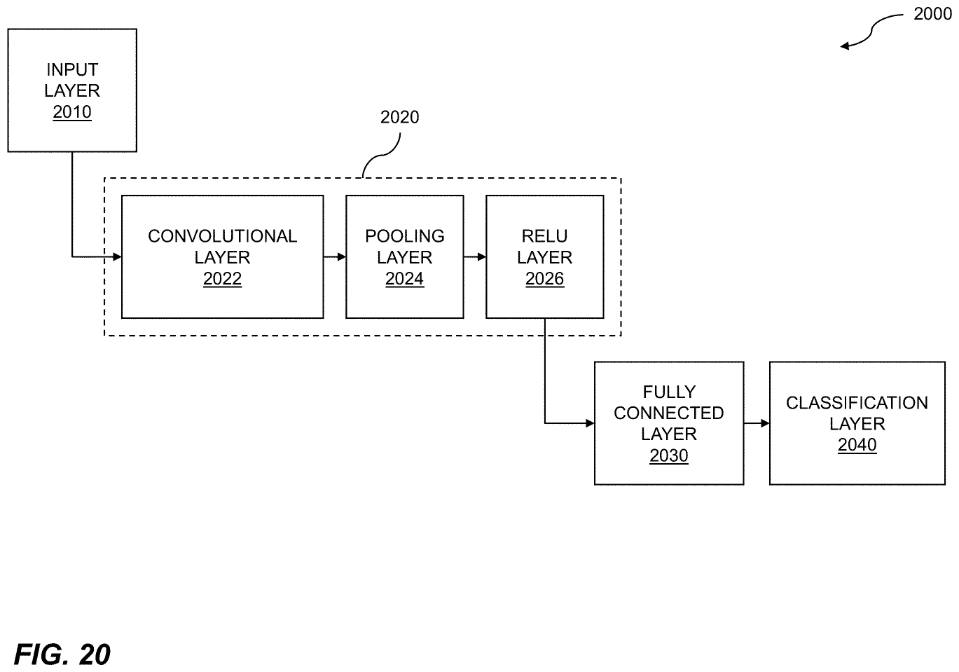

FIG. 20 is an example illustrating a convolutional neural network (CNN). A convolutional neural network such as the network 2000 can be used for deep learning, where the deep learning can be applied to distributed analysis for cognitive state metrics. Data for an individual is captured into a computing device. The data for the individual is uploaded to a web server. A cognitive state metric for the individual is calculated. Analysis from the web server is received by the computing device. The analysis is based on the data for the individual and the cognitive state metric for the individual. An output that describes a cognitive state of the individual is rendered at the computing device. The convolutional neural network analysis is output from the output layer. The convolutional neural network can be applied to such tasks as cognitive state analysis, mental state analysis, mood analysis, emotional state analysis, and so on. Cognitive state data can include mental processes, where the mental processes can include attention, creativity, memory, perception, problem solving, thinking, use of language, and the like.

Cognitive analysis is a very complex task. Understanding and evaluating moods, emotions, mental states, or cognitive states, requires a nuanced evaluation of facial expressions or other verbal and nonverbal cues that people generate. Cognitive state analysis is important in many areas such as research, psychology, business, intelligence, law enforcement, and so on. The understanding of cognitive states can be useful for a variety of business purposes such as improving marketing analysis, assessing the effectiveness of customer service interactions and retail experiences, and evaluating the consumption of content such as movies and videos. Identifying points of frustration in a customer transaction can allow a company to take action to address the causes of the frustration. By streamlining processes, key performance areas such as customer satisfaction and customer transaction throughput can be improved, resulting in increased sales and revenues. In a content scenario, producing compelling content that achieves the desired effect (e.g. fear, shock, laughter, etc.) can boost ticket sales and/or advertising revenue. If a movie studio is producing a horror movie, it is desirable to know if the scary scenes in the movie are achieving the desired effect. By conducting tests in sample audiences, and analyzing faces in the audience, a computer-implemented method and system can process thousands of faces to assess the cognitive state at the time of the scary scenes. In many ways, such an analysis can be more effective than surveys that ask audience members questions, since audience members may consciously or subconsciously change answers based on peer pressure or other factors. However, spontaneous facial expressions can be more difficult to conceal. Thus, by analyzing facial expressions en masse in real time, important information regarding the general cognitive state of the audience can be obtained.

Analysis of facial expressions is also a complex task. Image data, where the image data can include facial data, can be analyzed to identify a range of facial expressions. The facial expressions can include a smile, frown, smirk, and so on. The image data and facial data can be processed to identify the facial expressions. The processing can include analysis of expression data, action units, gestures, mental states, cognitive states, physiological data, and so on. Facial data as contained in the raw video data can include information on one or more of action units, head gestures, smiles, brow furrows, squints, lowered eyebrows, raised eyebrows, attention, and the like. The action units can be used to identify smiles, frowns, and other facial indicators of expressions. Gestures can also be identified, and can include a head tilt to the side, a forward lean, a smile, a frown, as well as many other gestures. Other types of data including physiological data can be collected, where the physiological data can be obtained using a camera or other image capture device, without contacting the person or persons. Respiration, heart rate, heart rate variability, perspiration, temperature, and other physiological indicators of cognitive state can be determined by analyzing the images and video data.

Deep learning is a branch of machine learning which seeks to imitate in software the activity which takes place in layers of neurons in the neocortex of the human brain. This imitative activity can enable software to “learn” to recognize and identify patterns in data, where the data can include digital forms of images, sounds, and so on. The deep learning software is used to simulate the large array of neurons of the neocortex. This simulated neocortex, or artificial neural network, can be implemented using mathematical formulas that are evaluated on processors. With the proliferating capabilities of the processors, increasing numbers of layers of the artificial neural network can be processed.

Deep learning applications include processing of image data, audio data, and so on. Image data applications include image recognition, facial recognition, etc. Image data applications can include differentiating dogs from cats, identifying different human faces, and the like. The image data applications can include identifying cognitive states, moods, mental states, emotional states, and so on, from the facial expressions of the faces that are identified. Audio data applications can include analyzing audio such as ambient room sounds, physiological sounds such as breathing or coughing, noises made by an individual such as tapping and drumming, voices, and so on. The voice data applications can include analyzing a voice for timbre, prosody, vocal register, vocal resonance, pitch, volume, speech rate, or language content. The voice data analysis can be used to determine one or more cognitive states, moods, mental states, emotional states, etc.

The artificial neural network, such as a convolutional neural network which forms the basis for deep learning, is based on layers. The layers can include an input layer, a convolutional layer, a fully connected layer, a classification layer, and so on. The input layer can receive input data such as image data, where the image data can include a variety of formats including pixel formats. The input layer can then perform processing tasks such as identifying boundaries of the face, identifying landmarks of the face, extracting features of the face, and/or rotating a face within the plurality of images. The convolutional layer can represent an artificial neural network such as a convolutional neural network. A convolutional neural network can contain a plurality of hidden layers. A convolutional layer can reduce the amount of data feeding into a fully connected layer. The fully connected layer processes each pixel/data point from the convolutional layer. A last layer within the multiple layers can provide output indicative of cognitive state. The last layer of the convolutional neural network can be the final classification layer. The output of the final classification layer can be indicative of the cognitive states of faces within the images that are provided to the input layer.

Deep networks including deep convolutional neural networks can be used for facial expression parsing. A first layer of the deep network includes multiple nodes, where each node represents a neuron within a neural network. The first layer can receive data from an input layer. The output of the first layer can feed to a second layer, where the latter layer also includes multiple nodes. A weight can be used to adjust the output of the first layer which is being input to the second layer. Some layers in the convolutional neural network can be hidden layers. The output of the second layer can feed to a third layer. The third layer can also include multiple nodes. A weight can adjust the output of the second layer which is being input to the third layer. The third layer may be a hidden layer. Outputs of a given layer can be fed to the next layer. Weights adjust the output of one layer as it is fed to the next layer. When the final layer is reached, the output of the final layer can be a facial expression, a cognitive state, a mental state, a characteristic of a voice, and so on. The facial expression can be identified using a hidden layer from the one or more hidden layers. The weights can be provided on inputs to the multiple layers to emphasize certain facial features within the face. The convolutional neural network can be trained to identify facial expressions, voice characteristics, etc. The training can include assigning weights to inputs on one or more layers within the multilayered analysis engine. One or more of the weights can be adjusted or updated during training. The assigning weights can be accomplished during a feed-forward pass through the multilayered neural network. In a feed-forward arrangement, the information moves forward from the input nodes, through the hidden nodes, and on to the output nodes. Additionally, the weights can be updated during a backpropagation process through the multilayered analysis engine.

Returning to the figure, FIG. 20 is an example showing a convolutional neural network 2000. The convolutional neural network can be used for deep learning, where the deep learning can be applied to avatar image animation using translation vectors. The deep learning system can be accomplished using a convolutional neural network or other techniques. The deep learning can perform facial recognition and analysis tasks. The network includes an input layer 2010. The input layer 2010 receives image data. The image data can be input in a variety of formats, such as JPEG, TIFF, BMP, and GIF. Compressed image formats can be decompressed into arrays of pixels, wherein each pixel can include an RGB tuple. The input layer 2010 can then perform processing such as identifying boundaries of the face, identifying landmarks of the face, extracting features of the face, and/or rotating a face within the plurality of images.

The network includes a collection of intermediate layers 2020. The multilayered analysis engine can include a convolutional neural network. Thus, the intermediate layers can include a convolutional layer 2022. The convolutional layer 2022 can include multiple sublayers, including hidden layers, within it. The output of the convolutional layer 2022 feeds into a pooling layer 2024. The pooling layer 2024 performs a data reduction, which makes the overall computation more efficient. Thus, the pooling layer reduces the spatial size of the image representation to reduce the number of parameters and computations in the network. In some embodiments, the pooling layer is implemented using filters of size 2×2, applied with a stride of two samples for every depth slice along both width and height, resulting in a reduction of 75-percent of the downstream node activations. The multilayered analysis engine can further include a max pooling layer as part of pooling layer 2024. Thus, in embodiments, the pooling layer is a max pooling layer, in which the output of the filters is based on a maximum of the inputs. For example, with a 2×2 filter, the output is based on a maximum value from the four input values. In other embodiments, the pooling layer is an average pooling layer or L2-norm pooling layer. Various other pooling schemes are possible.

The intermediate layers can include a Rectified Linear Units (RELU) layer 2026. The output of the pooling layer 2024 can be input to the RELU layer 2026. In embodiments, the RELU layer implements an activation function such as ƒ(x)−max(0,x), thus providing an activation with a threshold at zero. In some embodiments, the RELU layer 2026 is a leaky RELU layer. In this case, instead of the activation function providing zero when x<0, a small negative slope is used, resulting in an activation function such as ƒ(x)=1(x<0)(αx)+1(x>=0)(x). This can reduce the risk of “dying RELU” syndrome, where portions of the network can be “dead” with nodes/neurons that do not activate across the training dataset. The image analysis can comprise training a multilayered analysis engine using the plurality of images, wherein the multilayered analysis engine can comprise multiple layers that include one or more convolutional layers 2022 and one or more hidden layers, and wherein the multilayered analysis engine can be used for emotional analysis.

The example 2000 includes a fully connected layer 2030. The fully connected layer 2030 processes each pixel/data point from the output of the collection of intermediate layers 2020. The fully connected layer 2030 takes all neurons in the previous layer and connects them to every single neuron it has. The output of the fully connected layer 2030 provides input to a classification layer 2040. The output of the classification layer 2040 provides a facial expression and/or cognitive state. Thus, a multilayered analysis engine such as the one depicted in FIG. 20 processes image data using weights, models the way the human visual cortex performs object recognition and learning, and effectively analyzes image data to infer facial expressions and cognitive states.

Machine learning for generating parameters, analyzing data such as facial data and audio data, and so on, can be based on a variety of computational techniques. Generally, machine learning can be used for constructing algorithms and models. The constructed algorithms, when executed, can be used to make a range of predictions relating to data. The predictions can include whether an object in an image is a face, a box, or a puppy, whether a voice is female, male, or robotic, whether a message is legitimate email or a “spam” message, and so on. The data can include unstructured data and can be of large quantity. The algorithms that can be generated by machine learning techniques are particularly useful to data analysis because the instructions that comprise the data analysis technique do not need to be static. Instead, the machine learning algorithm or model, generated by the machine learning technique, can adapt. Adaptation of the learning algorithm can be based on a range of criteria such as success rate, failure rate, and so on. A successful algorithm is one that can adapt—or learn—as more data is presented to the algorithm. Initially, an algorithm can be “trained” by presenting it with a set of known data (supervised learning). Another approach, called unsupervised learning, can be used to identify trends and patterns within data. Unsupervised learning is not trained using known data prior to data analysis.

Reinforced learning is an approach to machine learning that is inspired by behaviorist psychology. The underlying premise of reinforced learning (also called reinforcement learning) is that software agents can take actions in an environment. The actions taken by the agents should maximize a goal such as a “cumulative reward”. A software agent is a computer program that acts on behalf of a user or other program. The software agent is implied to have the authority to act on behalf of the user or program. The actions taken are decided by action selection to determine what to do next. In machine learning, the environment in which the agents act can be formulated as a Markov decision process (MDP). The MDPs provide a mathematical framework for modeling of decision making in environments where the outcomes can be partly random (stochastic) and partly under the control of the decision maker. Dynamic programming techniques can be used for reinforced learning algorithms. Reinforced learning is different from supervised learning in that correct input/output pairs are not presented, and suboptimal actions are not explicitly corrected. Rather, online or computational performance is the focus. Online performance includes finding a balance between exploration of new (uncharted) territory or spaces and exploitation of current knowledge. That is, there is a tradeoff between exploration and exploitation.

Machine learning based on reinforced learning adjusts or learns based on learning an action, a combination of actions, and so on. An outcome results from taking an action. Thus, the learning model, algorithm, etc., learns from the outcomes that result from taking the action or combination of actions. The reinforced learning can include identifying positive outcomes, where the positive outcomes are used to adjust the learning models, algorithms, and so on. A positive outcome can be dependent on a context. When the outcome is based on a mood, emotional state, mental state, cognitive state, etc., of an individual, then a positive mood, emotional state, mental state, or cognitive state can be used to adjust the model and algorithm. Positive outcomes can include the person being more engaged, where engagement is based on affect, the person spending more time playing an online game or navigating a webpage, the person converting by buying a product or service, and so on. The reinforced learning can be based on exploring a solution space and adapting the model, algorithm, etc., based on outcomes of the exploration. When positive outcomes are encountered, the positive outcomes can be reinforced by changing weighting values within the model, algorithm, etc. Positive outcomes may result in increasing weighting values. Negative outcomes can also be considered, where weighting values may be reduced or otherwise adjusted.

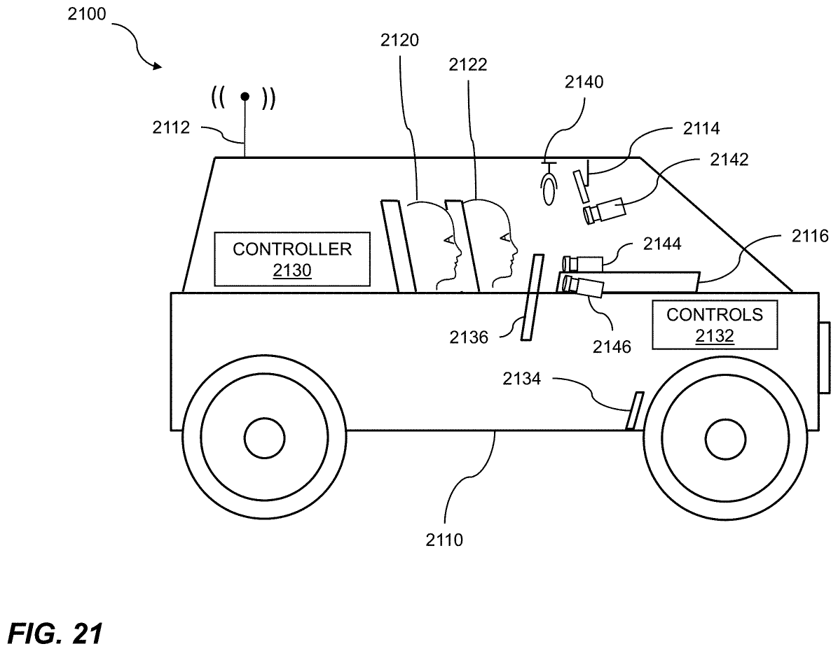

FIG. 21 is a system diagram for an interior of a vehicle 2100. A vehicle can be used in support of distributed analysis for cognitive state metrics. Data for an individual is captured into a computing device. The data for the individual is uploaded to a web server. A cognitive state metric for the individual is calculated. Analysis from the web server is received by the computing device. The analysis is based on the data for the individual and the cognitive state metric for the individual. An output is rendered at the computing device that describes a cognitive state of the individual. One or more occupants of a vehicle 2110, such as occupants 2120 and 2122, can be observed using a microphone 2140, one or more cameras 2142, 2144, or 2146, and other audio and image capture techniques. The image data can include video data. The video data and the audio data can include cognitive state data, where the cognitive state data can include facial data, voice data, physiological data, and the like. The occupant can be a driver occupant 2122 of the vehicle 2110, a passenger occupant 2120 within the vehicle, and so on.

The cameras or imaging devices that can be used to obtain images including facial data from the occupants of the vehicle 2110 can be positioned to capture the face of the vehicle operator, the face of a vehicle passenger, multiple views of the faces of occupants of the vehicle, and so on. The cameras can be located near a rear-view mirror 2114, such as camera 2142, positioned near or on a dashboard 2116, such as camera 2144, positioned within the dashboard, such as camera 2146, and so on. The microphone, or audio capture device, 2140 can be positioned within the vehicle such that voice data, speech data, non-speech vocalizations, and so on, can be easily collected with minimal background noise. In embodiments, additional cameras, imaging devices, microphones, audio capture devices, and so on, can be located throughout the vehicle. In further embodiments, each occupant of the vehicle could have multiple cameras, microphones, etc., positioned to capture video data and audio data from that occupant.

The interior of a vehicle 2110 can be a standard vehicle, an autonomous vehicle, a semi-autonomous vehicle, and so on. The vehicle can be a sedan or other automobile, a van, a sport utility vehicle (SUV), a truck, a bus, a special purpose vehicle, and the like. The interior of the vehicle 2110 can include standard controls such as a steering wheel 2136, a throttle control (not shown), a brake 2134, and so on. The interior of the vehicle can include other controls 2132 such as controls for seats, mirrors, climate adjustments, audio systems, etc. The controls 2132 of the vehicle 2110 can be controlled by a controller 2130. The controller 2130 can control the vehicle 2110 in various manners such as autonomously, semi-autonomously, assertively to a vehicle occupant 2120 or 2122, etc. In embodiments, the controller provides vehicle control techniques, assistance, etc. The controller 2130 can receive instructions via an antenna 2112 or using other wireless techniques. The controller 2130 can be preprogrammed to cause the vehicle to follow a specific route. The specific route that the vehicle is programmed to follow can be based on the cognitive state of the vehicle occupant. The specific route can be chosen based on lowest stress, least traffic, best view, shortest route, and so on.

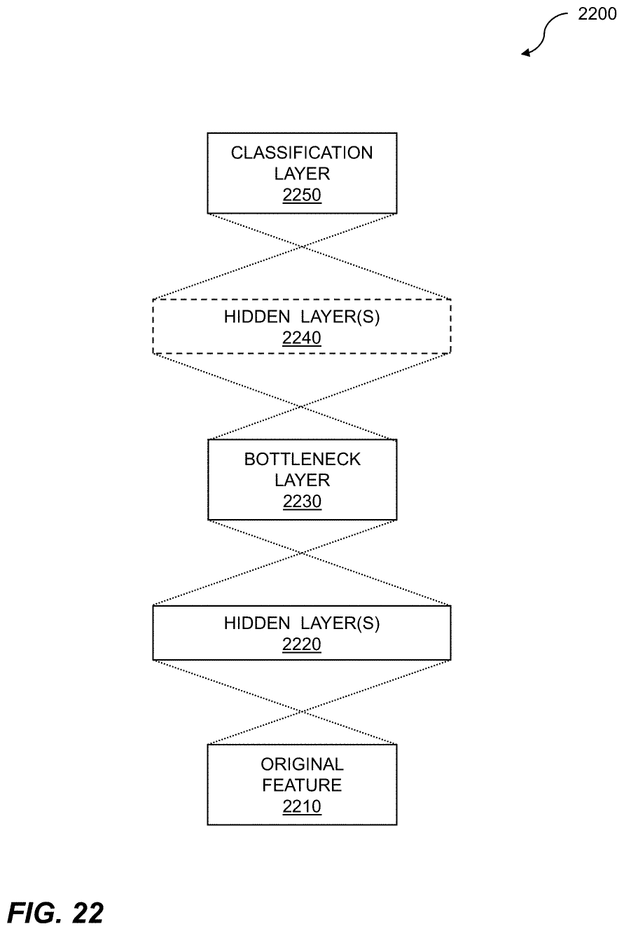

FIG. 22 illustrates a bottleneck layer within a deep learning environment. A plurality of layers in a deep neural network (DNN) can include a bottleneck layer. The deep neural network can comprise a convolutional neural network. The bottleneck layer can be used for distributed analysis for cognitive state metrics. A deep neural network can apply classifiers such as image classifiers, audio classifiers, and so on. The classifiers can be learned by analyzing cognitive state data. Data on a user interacting with a media presentation is collected at a client device. The data includes facial image data of the user. The facial image data is analyzed to extract cognitive state content of the user. One or more emotional intensity metrics are generated. The metrics are based on the cognitive state content. The media presentation is manipulated, based on the emotional intensity metrics and the cognitive state content.

Layers of a deep neural network can include a bottleneck layer 2200. A bottleneck layer can be used for a variety of applications such as facial recognition, voice recognition, emotional state recognition, and so on. The deep neural network in which the bottleneck layer is located can include a plurality of layers. The plurality of layers can include an original feature layer 2210. A feature such as an image feature can include points, edges, objects, boundaries between and among regions, properties, and so on. The deep neural network can include one or more hidden layers 2220. The one or more hidden layers can include nodes, where the nodes can include nonlinear activation functions and other techniques. The bottleneck layer can be a layer that learns translation vectors to transform a neutral face to an emotional or expressive face. In some embodiments, the translation vectors can transform a neutral voice to an emotional or expressive voice. Specifically, activations of the bottleneck layer determine how the transformation occurs. A single bottleneck layer can be trained to transform a neutral face or voice to a different emotional face or voice. In some cases, an individual bottleneck layer can be trained for a transformation pair. At runtime, once the user's emotion has been identified and an appropriate response to it can be determined (mirrored or complementary), the trained bottleneck layer can be used to perform the needed transformation.

The deep neural network can include a bottleneck layer 2230. The bottleneck layer can include a fewer number of nodes than the one or more preceding hidden layers. The bottleneck layer can create a constriction in the deep neural network or other network. The bottleneck layer can force information that is pertinent to a classification, for example, into a low dimensional representation. The bottleneck features can be extracted using an unsupervised technique. In other embodiments, the bottleneck features can be extracted using a supervised technique. The supervised technique can include training the deep neural network with a known dataset. The features can be extracted from an autoencoder such as a variational autoencoder, a generative autoencoder, and so on. The deep neural network can include hidden layers 2240. The number of the hidden layers can include zero hidden layers, one hidden layer, a plurality of hidden layers, and so on. The hidden layers following the bottleneck layer can include more nodes than the bottleneck layer. The deep neural network can include a classification layer 2250. The classification layer can be used to identify the points, edges, objects, boundaries, and so on, described above. The classification layer can be used to identify cognitive states, mental states, emotional states, moods, and the like. The output of the final classification layer can be indicative of the emotional states of faces within the images, where the images can be processed using the deep neural network.

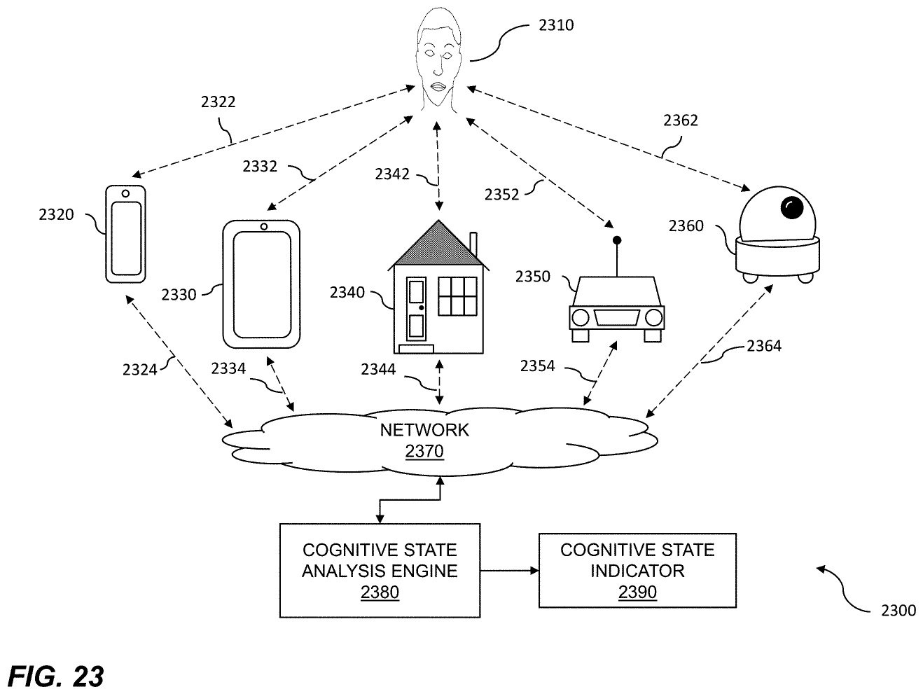

FIG. 23 shows data collection including multiple devices and locations 2300. One or more of the multiple devices and locations can enable distributed analysis for cognitive state metrics. Data for an individual is captured into a computing device. The data for the individual is uploaded to a web server. A cognitive state metric for the individual is calculated. Analysis from the web server is received by the computing device. The analysis is based on the data for the individual and the cognitive state metric for the individual. An output is rendered at the computing device that describes a cognitive state of the individual.

The multiple mobile devices, vehicles, and locations 2300 can be used separately or in combination to collect video data on a user 2310. The video data can include facial data, image data, etc. Other data such as audio data, physiological data, and so on, can be collected on the user. While one person is shown, the video data, or other data, can be collected on multiple people. A user 2310 can be observed as she or he is performing a task, experiencing an event, viewing a media presentation, and so on. The user 2310 can be shown one or more media presentations, political presentations, social media, or another form of displayed media. The one or more media presentations can be shown to a plurality of people. The media presentations can be displayed on an electronic display coupled to a client device. The data collected on the user 2310 or on a plurality of users can be in the form of one or more videos, video frames, still images, etc. The plurality of videos can be of people who are experiencing different situations. Some example situations can include the user or plurality of users being exposed to TV programs, movies, video clips, social media, social sharing, and other such media. The situations could also include exposure to media such as advertisements, political messages, news programs, and so on. As noted before, video data can be collected on one or more users in substantially identical or different situations and viewing either a single media presentation or a plurality of presentations. The data collected on the user 2310 can be analyzed and viewed for a variety of purposes including expression analysis, mental state analysis, cognitive state analysis, and so on. The electronic display can be on a smartphone 2320 as shown, a tablet computer 2330, a personal digital assistant, a television, a mobile monitor, or any other type of electronic device. In one embodiment, expression data is collected on a mobile device such as a cell phone 2320, a tablet computer 2330, a laptop computer, or a watch. Thus, the multiple sources can include at least one mobile device, such as a phone 2320 or a tablet 2330, or a wearable device such as a watch or glasses (not shown). A mobile device can include a front-facing camera and/or a rear-facing camera that can be used to collect expression data. Sources of expression data can include a webcam, a phone camera, a tablet camera, a wearable camera, and a mobile camera. A wearable camera can comprise various camera devices, such as a watch camera. In addition to using client devices for data collection from the user 2310, data can be collected in a house 2340 using a web camera or the like; in a vehicle 2350 using a web camera, client device, etc.; by a social robot 2360, and so on.

As the user 2310 is monitored, the user 2310 might move due to the nature of the task, boredom, discomfort, distractions, or for another reason. As the user moves, the camera with a view of the user's face can be changed. Thus, as an example, if the user 2310 is looking in a first direction, the line of sight 2322 from the smartphone 2320 is able to observe the user's face, but if the user is looking in a second direction, the line of sight 2332 from the tablet 2330 is able to observe the user's face. Furthermore, in other embodiments, if the user is looking in a third direction, the line of sight 2342 from a camera in the house 2340 is able to observe the user's face, and if the user is looking in a fourth direction, the line of sight 2352 from the camera in the vehicle 2350 is able to observe the user's face. If the user is looking in a fifth direction, the line of sight 2362 from the social robot 2360 is able to observe the user's face. If the user is looking in a sixth direction, a line of sight from a wearable watch-type device, with a camera included on the device, is able to observe the user's face. In other embodiments, the wearable device is another device, such as an earpiece with a camera, a helmet or hat with a camera, a clip-on camera attached to clothing, or any other type of wearable device with a camera or other sensor for collecting expression data. The user 2310 can also use a wearable device including a camera for gathering contextual information and/or collecting expression data on other users. Because the user 2310 can move her or his head, the facial data can be collected intermittently when she or he is looking in a direction of a camera. In some cases, multiple people can be included in the view from one or more cameras, and some embodiments include filtering out faces of one or more other people to determine whether the user 2310 is looking toward a camera. All or some of the expression data can be continuously or sporadically available from the various devices and other devices.

The captured video data can include cognitive content, such as facial expressions, etc., and can be transferred over a network 2370. The network can include the Internet or other computer network. The smartphone 2320 can share video using a link 2324, the tablet 2330 using a link 2334, the house 2340 using a link 2344, the vehicle 2350 using a link 2354, and the social robot 2360 using a link 2364. The links 2324, 2334, 2344, 2354, and 2364 can be wired, wireless, and hybrid links. The captured video data, including facial expressions, can be analyzed on a cognitive state analysis engine 2380, on a computing device such as the video capture device, or on another separate device. The analysis could take place on one of the mobile devices discussed above, on a local server, on a remote server, and so on. In embodiments, some of the analysis takes place on the mobile device, while other analysis takes place on a server device. The analysis of the video data can include the use of a classifier. The video data can be captured using one of the mobile devices discussed above and sent to a server or another computing device for analysis. However, the captured video data including expressions can also be analyzed on the device which performed the capturing. The analysis can be performed on a mobile device where the videos were obtained with the mobile device and wherein the mobile device includes one or more of a laptop computer, a tablet, a PDA, a smartphone, a wearable device, and so on. In another embodiment, the analyzing comprises using a classifier on a server or another computing device different from the capture device. The analysis data from the cognitive state analysis engine can be processed by a cognitive state indicator 2390. The cognitive state indicator 2390 can indicate cognitive states, mental states, moods, emotions, etc. Further embodiments include inferring a cognitive state based on emotional content within a face detected within the facial image data, wherein the cognitive state includes of one or more of drowsiness, fatigue, distraction, impairment, sadness, stress, happiness, anger, frustration, confusion, disappointment, hesitation, cognitive overload, focusing, engagement, attention, boredom, exploration, confidence, trust, delight, disgust, skepticism, doubt, satisfaction, excitement, laughter, calmness, curiosity, humor, depression, envy, sympathy, embarrassment, poignancy, or mirth.

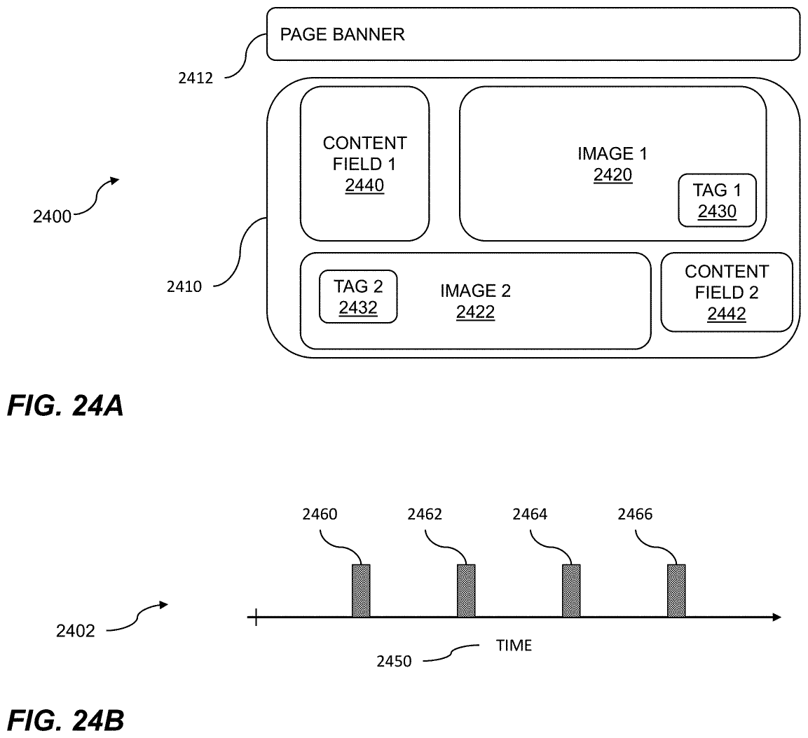

FIG. 24A shows example tags embedded in a webpage. A webpage 2400 can include a page body 2410, a page banner 2412, and so on. The page body can include one or more objects, where the objects can include text, images, videos, audio, and so on. The example page body 2410 shown includes a first image, image 1 2420; a second image, image 2 2422; a first content field, content field 1 2440; and a second content field, content field 2 2442. In practice, the page body 2410 can contain any number of images and content fields and can include one or more videos, one or more audio presentations, and so on. The page body can include embedded tags, such as tag 1 2430 and tag 2 2432. In the example shown, tag 1 2430 is embedded in image 1 2420, and tag 2 2432 is embedded in image 2 2422. In embodiments, any number of tags is embedded. Tags can also be embedded in content fields, in videos, in audio presentations, etc. When a user mouses over a tag or clicks on an object associated with a tag, the tag can be invoked. For example, when the user mouses over tag 1 2430, tag 1 2430 can then be invoked. Invoking tag 1 2430 can include enabling a camera coupled to a user's device and capturing one or more images of the user as the user views a media presentation (or digital experience). In a similar manner, when the user mouses over tag 2 2432, tag 2 2432 can be invoked. Invoking tag 2 2432 can also include enabling the camera and capturing images of the user. In other embodiments, other actions are taken based on invocation of the one or more tags. For example, invoking an embedded tag can initiate an analysis technique, post to social media, award the user a coupon or another prize, initiate cognitive state analysis, perform emotion analysis, and so on.

FIG. 24B shows example tag invoking for the collection of images. As stated above, a media presentation can be a video, a webpage, and so on. A video 2402 can include one or more embedded tags, such as a tag 2460, another tag 2462, a third tag 2464, a fourth tag 2466, and so on. In practice, any number of tags can be included in the media presentation. The one or more tags can be invoked during the media presentation. The collection of the invoked tags can occur over time as represented by a timeline 2450. When a tag is encountered in the media presentation, the tag can be invoked. For example, when the tag 2460 is encountered, invoking the tag can enable a camera coupled to a user's device and can capture one or more images of the user viewing the media presentation. Invoking a tag can depend on opt-in by the user. For example, if a user has agreed to participate in a study by indicating an opt-in, then the camera coupled to the user's device can be enabled and one or more images of the user can be captured. If the user has not agreed to participate in the study and has not indicated an opt-in, then invoking the tag 2460 does not enable the camera nor capture images of the user during the media presentation. The user can indicate an opt-in for certain types of participation, where opting-in can be dependent on specific content in the media presentation. For example, the user could opt in to participation in a study of political campaign messages and not opt in for a particular advertisement study. In this case, tags that are related to political campaign messages and that enable the camera and image capture when invoked would be embedded in the media presentation. However, tags imbedded in the media presentation that are related to advertisements would not enable the camera when invoked. Various other situations of tag invocation are possible.

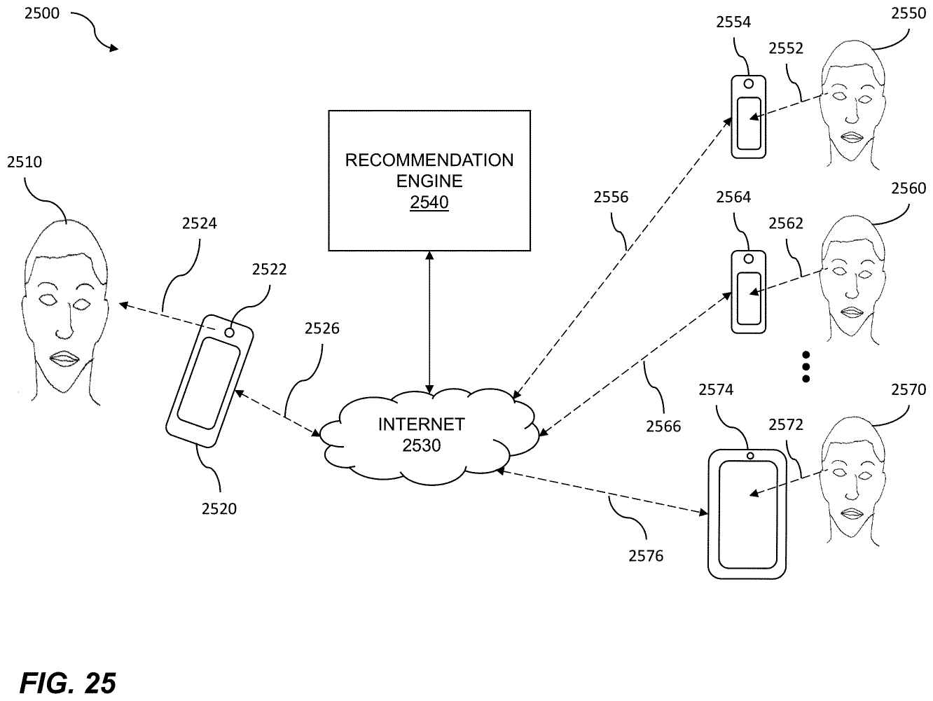

FIG. 25 shows an example livestreaming social video scenario. Livestreaming video is an example of one-to-many social media where video can be sent over the Internet from one person to a plurality of people using a social media app and/or platform. Livestreaming is one of numerous popular techniques used by people who want to disseminate ideas, send information, provide entertainment, share experiences, and so on. Some of the livestreams can be scheduled, such as webcasts, online classes, sporting events, news, computer gaming, or video conferences, while others can be impromptu streams that are broadcast as and when needed or desirable. Examples of impromptu livestream videos can range from individuals simply wanting to share experiences with their social media followers, to coverage of breaking news, emergencies, or natural disasters. This latter coverage is known as mobile journalism or “mo jo” and is becoming increasingly commonplace. “Reporters” can use networked, portable electronic devices to provide mobile journalism content to a plurality of social media followers. Such reporters can be quickly and inexpensively deployed as the need or desire arises.

Several livestreaming social media apps and platforms can be used for transmitting video. One such video social media app is Meerkat™ that can link with a user's Twitter™ account. Meerkat™ enables a user to stream video using a handheld, networked, electronic device coupled to video capabilities. Viewers of the livestream can comment on the stream using tweets that can be seen by and responded to by the broadcaster. Another popular app is Periscope™ that can transmit a live recording from one user to that user's Periscope™ or other social media followers. The Periscope™ app can be executed on a mobile device. The user's followers can receive an alert whenever that user begins a video transmission. Another livestream video platform is Twitch which can be used for video streaming of video gaming, and broadcasts of various competitions, concerts and other events.

The example 2500 shows user 2510 broadcasting a video livestream to one or more people 2550, 2560, 2570, and so on. A portable, network-enabled electronic device 2520 can be coupled to a front-side camera 2522. The portable electronic device 2520 can be a smartphone, a PDA, a tablet, a laptop computer, and so on. The camera 2522 coupled to the device 2520 can have a line-of-sight view 2524 to the user 2510 and can capture video of the user 2510. The captured video can be sent to a recommendation engine 2540 using a network link 2526 to the Internet 2530. The network link can be a wireless link, a wired link, and so on. The recommendation engine 2540 can recommend to the user 2510 an app and/or platform that can be supported by the server and can be used to provide a video live-stream to one or more followers of the user 2510. The example 2500 shows three followers of the user 2510, followers 2550, 2560, and 2570. Each follower has a line-of-sight view to a video screen on a portable, networked electronic device. In other embodiments, one or more followers follow the user 2510 using any other networked electronic device, including a computer. In the example 2500, the person 2550 has a line-of-sight view 2552 to the video screen of a device 2554, the person 2560 has a line-of-sight view 2562 to the video screen of a device 2564, and the person 2570 has a line-of-sight view 2572 to the video screen of a device 2574. The portable electronic devices 2554, 2564, and 2574 each can be a smartphone, a PDA, a tablet, and so on. Each portable device can receive the video stream being broadcast by the user 2510 through the Internet 2530 using the app and/or platform that can be recommended by the recommendation engine 2540. The device 2554 can receive a video stream using the network link 2556, the device 2564 can receive a video stream using the network link 2566, the device 2574 can receive a video stream using the network link 2576, and so on. The network link can be a wireless link, and wired link, and so on. Depending on the app and/or platform that can be recommended by the recommendation engine 2540, one or more followers, such as the followers 2550, 2560, 2570, and so on, can reply to, comment on, and otherwise provide feedback to the user 2510 using their devices 2554, 2564, and 2574 respectively.

As described above, one or more videos of various types, including livestreamed videos, can be presented to a plurality of users for wide ranging purposes. These purposes can include, but are not limited to, entertainment, education, general information, political campaign messages, social media sharing, and so on. Cognitive state data can be collected from the one or more users as they view the videos. The collection of the cognitive state data can be based on a user agreeing to enable a camera that can be used for the collection of the cognitive state data. The collected cognitive state data can be analyzed for various purposes. When the cognitive state data has been collected from a sufficient number of users to enable anonymity, then the aggregated cognitive state data can be used to provide information on aggregated cognitive states of the viewers. The aggregated cognitive states can be used to recommend videos that can include media presentations, for example. The recommendations of videos can be based on videos that can be similar to those videos to which a user had a particular cognitive state response, for example. The recommendations of videos can include videos to which the user can be more likely to have a favorable cognitive state response, videos that can be enjoyed by the user's social media contacts, videos that can be trending, and so on.

The aggregated cognitive state data can be represented using a variety of techniques and can be presented to the one or more users. The aggregated cognitive state data can be presented while the one or more users are viewing the video, and the aggregated cognitive state data can be presented after the one or more users have viewed the video. The video can be obtained from a server, a collection of videos, a livestream video, and so on. The aggregated cognitive state data can be presented to the users using a variety of techniques. For example, the aggregated cognitive state data can be displayed as colored dots, as graphs, etc. The colored dots, graphs, and so on, can be displayed with the video, embedded in the video, viewed subsequently to viewing the video, or presented in another fashion. The aggregated cognitive state data can also be used to provide feedback to the originator of the video, where the feedback can include viewer reaction or reactions to the video, receptiveness to the video, effectiveness of the video, etc. The aggregated cognitive state data can include sadness, happiness, frustration, confusion, disappointment, hesitation, cognitive overload, focusing, being engaged, attending, boredom, exploration, confidence, trust, delight, valence, skepticism, satisfaction, and so on. The videos can include livestreamed videos. The videos and the livestreamed videos can be presented along with the aggregated cognitive state data from the one or more users. The aggregated cognitive state data, as viewed by the users, can be employed by the same users to determine what cognitive states are being experienced by other users as all parties view a given video, when those cognitive states occur, whether those cognitive states are similar to the one or more cognitive states experienced by the users, and so on. The viewing of the aggregated cognitive state data can enable a viewer to experience videos viewed by others, to feel connected to other users who are viewing the videos, to share in the experience of viewing the videos, to gauge the cognitive states experienced by the users, and so on.

The collecting of cognitive state data can be performed as one or more users observe the videos described above. For example, a news site, a social media site, a crowdsourced site, an individual's digital electronic device, and so on can provide the videos. The cognitive state data can be collected as the one or more users view a given video or livestream video. The cognitive state data can be recorded and analyzed. The results of the analysis of the collected cognitive state data from the one or more users can be displayed to the one or more users following the viewing of the video, for example. For confidentiality reasons, cognitive state data can be collected from a minimum or threshold number of users before the aggregated cognitive state data is displayed. One or more users on one or more social media sites can share their individual cognitive state data and the aggregated cognitive state data that can be collected. For example, a user could share with their Facebook™ friends her or his cognitive state data results from viewing a particular video. How a user responds to a video can be compared to the responses of their friends, of other users, and so on, using a variety of techniques including a social graph. For example, the user could track the reactions of her or his friends to a particular video using a Facebook™ social graph. The cognitive state data can be shared automatically or can be shared manually, as selected by the user. Automatic sharing of cognitive state data can be based on user credentials such as logging in to a social media site. A user's privacy can also be enabled using a variety of techniques, including anonymizing a user's cognitive state data, anonymizing and/or deleting a user's facial data, and so on. Facial tracking data can be provided in real time. In embodiments, the user has full control of playback of a video, a streamed video, a livestreamed video, and so on. That is, the user can pause, skip, scrub, go back, stop, and so on. Recommendations can be made to the user regarding viewing another video. The flow of a user viewing a video can continue from the current video to another video based on the recommendations. The next video can be a streamed video, a livestreamed video, and so on.

In another embodiment, aggregated cognitive state data can be used to assist a user to select a video, video stream, livestream video, and so on, that can be considered most engaging to the user. By way of example, if there is a user who is interested in a particular type of video stream such as a gaming stream, a sports stream, a news stream, a movie stream, and so on, and that favorite video stream is not currently available to the user, then recommendations can be made to the user based on a variety of criteria to assist in finding an engaging video stream. For example, the user can connect to a video stream that is presenting one or more sports events, but if the stream does not include the stream of the user's favorite, then recommendations can be made to the user based on aggregated cognitive state data of other users who are ranking or reacting to the one or more sports events currently available. Similarly, if analysis of the cognitive state data collected from the user indicates that the user is not reacting favorably to a given video stream, then a recommendation can be made for another video stream based on an audience who is engaged with the latter stream.

A given user can choose to participate in collection of cognitive state data for a variety of purposes. One or more personae can be used to characterize or classify a given user who views one or more videos. The personae can be useful for recommending one or more videos to a user based on cognitive state data collected from the user, for example. The recommending of one or more videos to the user can be based on aggregated cognitive state data collected from one or more users with a similar persona. Many personae can be described and chosen based on a variety of criteria. For example, personae can include a demo user, a social sharer, a video viewing enthusiast, a viral video enthusiast, an analytics researcher, a quantified self-user, a music aficionado, and so on. Any number of personae can be described, and any number of personae can be assigned to a particular user.

A demo user can be a user who is curious about the collection of cognitive state data and the presentation of that cognitive state data. The demo user can view any number of videos in order to experience the cognitive state data collection and to observe their own social curve, for example. The demo user can view some viral videos in order to observe an aggregated population. The demo user can be interested in trying cognitive state data collection and presentation in order to determine how she or he would use such a technique for their own purposes.

A social sharer can be a user who is enthusiastic about sharing demos and videos with their friends. The friends can be social media friends such as Facebook™ friends, for example. The videos can be particularly engaging, flashy, slickly produced, and so on. The social sharer can be interested in the reactions to and the sharing of the video that the social sharer has shared. The social sharer can also compare their own cognitive states to those of their friends. The social sharer can use the comparison to increase their knowledge of their friends and to gather information about the videos that those friends enjoyed.

A video-viewing enthusiast can be a user who enjoys watching videos and desires to watch more videos. Such a persona can generally stay within the context of a video streaming site, for example. The viewing by the user can be influenced by recommendations that can draw the user back to view more videos. When the user finds that the recommendations are desirable, then the user will likely continue watching videos within the streaming site. The video enthusiast can want to find the videos that the user wants to watch and also the portions of the videos that the user wants to watch.

A viral video enthusiast can be a user who chooses to watch many videos through social media. The social media can include links, shares, comments, etc. from friends of the user, for example. When the user clicks on the link to the video, the user can be connected from the external site to the video site. For example, the user can click a link in Reddit™, Twitter™, Facebook™, etc. and be connected to a video on YouTube™ or another video sharing site. Such a user is interested in seamless integration between the link on the social media site and the playing of the video on the video streaming site. The video streaming site can be a livestreaming video site.

An analytics researcher or “uploader” can be a user who can be interested in tracking video performance of one or more videos over time. The performance of the one or more videos can be based on various metrics, including emotional engagement of one or more viewers as they view the one or more videos. The analytics researcher can be interested primarily in the various metrics that can be generated based on a given video. The analytics can be based on demographic data, geographic data, and so on. Analytics can also be based on trending search terms, popular search terms, and so on, where the search terms can be identified using web facilities such as Google Trends™.

A quantified self-user can be a user who can be interested in studying and/or documenting her or his own video watching experiences. The qualified self-user reviews her or his cognitive state data over time, can sort a list of viewed videos over a time period, and so on. The qualified self-user can compare their cognitive state data that is collected while watching a given video with their personal norms. This user persona can also provide feedback. The quantified self-user can track their reactions to one or more videos over time and over videos, where tracking over videos can include tracking favorite videos, categorizing videos that have been viewed, remembering favorite videos, etc.

A music enthusiast can be a user who is a consumer of music and who uses a video streaming site such as a music streaming site. For example, this user persona can use music mixes from sites such as YouTube™ as if they were provided by a music streaming site such as Spotify™, Pandora™, Apple Music™, Tidal™, and so on. The music enthusiast persona can be less likely to be sitting in front of a screen, since their main mode of engagement is sound rather than sight. Facial reactions that can be captured from the listener can be weaker, for example, than those facial reactions captured from a viewer.

The method can include comparing the cognitive state data that was captured against cognitive state event temporal signatures. In embodiments, the method includes identifying a cognitive state event type based on the comparing. The recommending of the second media presentation can be based on the cognitive state event type. The recommending of the second media presentation can be performed using one or more processors. The first media presentation can include a first socially shared livestream video. The method can further comprise generating highlights for the first socially shared livestream video, based on the cognitive state data that was captured. The first socially shared livestream video can include an overlay with information on the cognitive state data that was captured. The overlay can include information on the cognitive state data collected from the other people. The cognitive state data that was captured for the first socially shared livestream video can be analyzed substantially in real time. In some embodiments, the second media presentation includes a second socially shared livestream video. The method can further comprise a recommendation for changing from the first socially shared livestream video to the second socially shared livestream video. The first socially shared livestream video can be broadcast to a plurality of people. In embodiments, the method further comprises providing an indication to the individual that the second socially shared livestream video is ready to be joined.

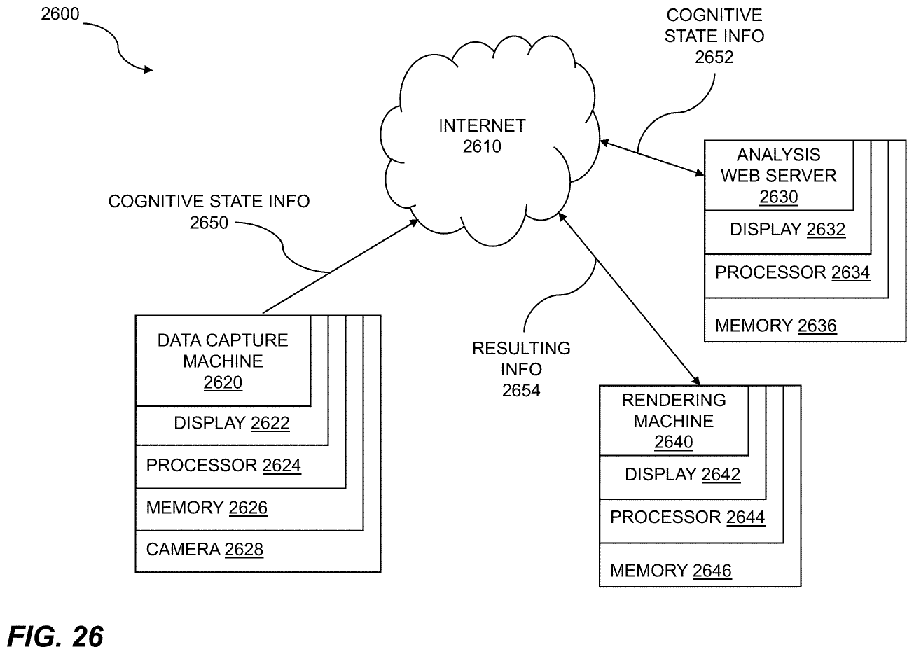

FIG. 26 is a system diagram for cognitive state metric analysis. The cognitive state metric analysis can include analyzing cognitive state and emotional content from data captured for an individual or a plurality of individuals. The system 2600 can be implemented using one or more machines. The system 2600 includes aspects of cognitive data capture, calculation and analysis, and rendering. The system 2600 can include a memory which stores instructions and one or more processors coupled to the memory wherein the one or more processors, when executing the instructions which are stored, are configured to: capture data for an individual into a computing device, wherein the data provides information for evaluating a cognitive state of the individual; upload the data for the individual to a web server; calculate a cognitive state metric for the individual, on the web server, based on the data that was uploaded; receive analysis from the web server, by the computing device, wherein the analysis is based on the data for the individual and the cognitive state metric for the individual; and render an output at the computing device that describes a cognitive state of the individual, based on the analysis that was received. The system 2600 can perform a computer-implemented method for distributed analysis comprising: capturing data for an individual into a computing device, wherein the data provides information for evaluating a cognitive state of the individual; uploading the data for the individual to a web server; calculating a cognitive state metric for the individual, on the web server, based on the data that was uploaded; receiving analysis from the web server, by the computing device, wherein the analysis is based on the data for the individual and the cognitive state metric for the individual; and rendering an output at the computing device that describes a cognitive state of the individual, based on the analysis that was received.

The system 2600 can include one or more data collection machines 2620 linked to an analysis web server 2630 and a rendering machine 2640 via the Internet 2610 or another computer network. The network can be wired or wireless, a combination of wired and wireless networks, and so on. Cognitive state information 2650 and 2652 can be transferred to the analysis server 2630 through the Internet 2610, for example. The example data capture machine 2620 shown comprises one or more processors 2624 coupled to a memory 2626 which can store and retrieve instructions, a display 2622, and a camera 2628. The camera 2628 can include a webcam, a video camera, a still camera, a thermal imager, a CCD device, a phone camera, a three-dimensional camera, a depth camera, a light field camera, a plenoptic camera, multiple webcams used to show different views of a person, or any other type of image capture technique that can allow captured data to be used in an electronic system. The memory 2626 can be used for storing instructions, data on a plurality of people, gaming data, one or more classifiers, one or more actions units, and so on. The display 2622 can be any electronic display, including but not limited to, a computer display, a laptop screen, a netbook screen, a tablet computer screen, a smartphone display, a mobile device display, a remote with a display, a television, a projector, or the like. Cognitive state information 2650 can be transferred via the Internet 2610 for a variety of purposes including analysis, calculation, rendering, storage, cloud storage, sharing, social sharing, and so on.

The analysis server 2630 can include one or more processors 2634 coupled to a memory 2636 which can store and retrieve instructions, and it can also include a display 2632. The analysis server 2630 can receive analytics for livestreaming as well as cognitive state information 2652 and can analyze the information using classifiers, action units, and so on. The classifiers and action units can be stored in the analysis server, loaded into the analysis server, provided by a user of the analysis server, and so on. The analysis server 2630 can use image data received from the data capture machine 2620 to produce resulting information 2654. The resulting information can include an emotion, a mood, a cognitive state, etc., and can also be based on the analytics for livestreaming. In some embodiments, the analysis server 2630 receives data from a plurality of data capture machines, aggregates the data, processes the data or the aggregated data, and so on.

The rendering machine 2640 can include one or more processors 2644 coupled to a memory 2646 which can store and retrieve instructions and data, and it can also include a display 2642. The rendering of the resulting information rendering data 2654 can occur on the rendering machine 2640 or on a different platform from the rendering machine 2640. In embodiments, the rendering of the resulting information rendering data 2654 occurs on the image data collection machine 2620 or on the analysis server 2630. As shown in the system 2600, the rendering machine 2640 can receive resulting information rendering data 2654 via the Internet 2610 or another network from the data capture machine 2620, from the analysis web server 2630, or from both. The rendering can include a visual display or any other appropriate display format. In embodiments, the data capture machine 2620 and the rendering machine 2640 are the same machine.

The system 2600 can include a computer program product stored on a non-transitory computer-readable medium for distributed analysis, the computer program product comprising code which causes one or more processors to perform operations of: capturing data for an individual into a computing device, wherein the data provides information for evaluating a cognitive state of the individual; uploading the data for the individual to a web server; calculating a cognitive state metric for the individual, on the web server, based on the data that was uploaded; receiving analysis from the web server, by the computing device, wherein the analysis is based on the data for the individual and the cognitive state metric for the individual; and rendering an output at the computing device that describes a cognitive state of the individual, based on the analysis that was received.

Each of the above methods may be executed on one or more processors on one or more computer systems. Embodiments may include various forms of distributed computing, client/server computing, and cloud-based computing. Further, it will be understood that the depicted steps or boxes contained in this disclosure's flow charts are solely illustrative and explanatory. The steps may be modified, omitted, repeated, or re-ordered without departing from the scope of this disclosure. Further, each step may contain one or more sub-steps. While the foregoing drawings and description set forth functional aspects of the disclosed systems, no particular implementation or arrangement of software and/or hardware should be inferred from these descriptions unless explicitly stated or otherwise clear from the context. All such arrangements of software and/or hardware are intended to fall within the scope of this disclosure.

The block diagrams and flowchart illustrations depict methods, apparatus, systems, and computer program products. The elements and combinations of elements in the block diagrams and flow diagrams, show functions, steps, or groups of steps of the methods, apparatus, systems, computer program products and/or computer-implemented methods. Any and all such functions—generally referred to herein as a “circuit,” “module,” or “system”—may be implemented by computer program instructions, by special-purpose hardware-based computer systems, by combinations of special purpose hardware and computer instructions, by combinations of general purpose hardware and computer instructions, and so on.

A programmable apparatus which executes any of the above-mentioned computer program products or computer-implemented methods may include one or more microprocessors, microcontrollers, embedded microcontrollers, programmable digital signal processors, programmable devices, programmable gate arrays, programmable array logic, memory devices, application specific integrated circuits, or the like. Each may be suitably employed or configured to process computer program instructions, execute computer logic, store computer data, and so on.

It will be understood that a computer may include a computer program product from a computer-readable storage medium and that this medium may be internal or external, removable and replaceable, or fixed. In addition, a computer may include a Basic Input/Output System (BIOS), firmware, an operating system, a database, or the like that may include, interface with, or support the software and hardware described herein.

Embodiments of the present invention are neither limited to conventional computer applications nor the programmable apparatus that run them. To illustrate: the embodiments of the presently claimed invention could include an optical computer, quantum computer, analog computer, or the like. A computer program may be loaded onto a computer to produce a particular machine that may perform any and all of the depicted functions. This particular machine provides a means for carrying out any and all of the depicted functions.

Any combination of one or more computer readable media may be utilized including but not limited to: a non-transitory computer readable medium for storage; an electronic, magnetic, optical, electromagnetic, infrared, or semiconductor computer readable storage medium or any suitable combination of the foregoing; a portable computer diskette; a hard disk; a random access memory (RAM); a read-only memory (ROM), an erasable programmable read-only memory (EPROM, Flash, MRAM, FeRAM, or phase change memory); an optical fiber; a portable compact disc; an optical storage device; a magnetic storage device; or any suitable combination of the foregoing. In the context of this document, a computer readable storage medium may be any tangible medium that can contain or store a program for use by or in connection with an instruction execution system, apparatus, or device.

It will be appreciated that computer program instructions may include computer executable code. A variety of languages for expressing computer program instructions may include without limitation C, C++, Java, JavaScript™, ActionScript™, assembly language, Lisp, Perl, Tcl, Python, Ruby, hardware description languages, database programming languages, functional programming languages, imperative programming languages, and so on. In embodiments, computer program instructions may be stored, compiled, or interpreted to run on a computer, a programmable data processing apparatus, a heterogeneous combination of processors or processor architectures, and so on. Without limitation, embodiments of the present invention may take the form of web-based computer software, which includes client/server software, software-as-a-service, peer-to-peer software, or the like.

In embodiments, a computer may enable execution of computer program instructions including multiple programs or threads. The multiple programs or threads may be processed approximately simultaneously to enhance utilization of the processor and to facilitate substantially simultaneous functions. By way of implementation, any and all methods, program codes, program instructions, and the like described herein may be implemented in one or more threads which may in turn spawn other threads, which may themselves have priorities associated with them. In some embodiments, a computer may process these threads based on priority or other order.

Unless explicitly stated or otherwise clear from the context, the verbs “execute” and “process” may be used interchangeably to indicate execute, process, interpret, compile, assemble, link, load, or a combination of the foregoing. Therefore, embodiments that execute or process computer program instructions, computer-executable code, or the like may act upon the instructions or code in any and all of the ways described. Further, the method steps shown are intended to include any suitable method of causing one or more parties or entities to perform the steps. The parties performing a step, or portion of a step, need not be located within a particular geographic location or country boundary. For instance, if an entity located within the United States causes a method step, or portion thereof, to be performed outside of the United States then the method is considered to be performed in the United States by virtue of the causal entity.

While the invention has been disclosed in connection with preferred embodiments shown and described in detail, various modifications and improvements thereon will become apparent to those skilled in the art. Accordingly, the foregoing examples should not limit the spirit and scope of the present invention; rather it should be understood in the broadest sense allowable by law.

What is claimed is:

1. A computer implemented method for distributed analysis comprising:
capturing data for an individual into a computing device, wherein the data provides information for evaluating a cognitive state of the individual;
uploading the data for the individual to a web server;
calculating a cognitive state metric for the individual, on the web server, based on the data that was uploaded;
receiving analysis from the web server, by the computing device, wherein the analysis is based on the data for the individual and the cognitive state metric for the individual; and
rendering an output at the computing device that describes a cognitive state of the individual, based on the analysis that was received.

2. The method of claim 1 wherein the cognitive state metric is based on a facial expression metric for the individual.

3. The method of claim 2 wherein the facial expression metric for the individual is calculated on facial image data captured as part of the data for the individual.

4. The method of claim 3 wherein the calculation on facial image data is performed on the web server.

5. The method of claim 3 wherein the calculation on facial image data is performed on the computing device before uploading to the web server.

6. The method of claim 1 further comprising including an emotional intensity metric in the cognitive state metric.

7. The method of claim 1 wherein the analysis includes demographic information distilled from the data.

8. The method of claim 1 further comprising capturing further data for a second individual.

9. The method of claim 8 further comprising determining weights and image classifiers, wherein the determining is performed on a remote server, based on the data for the individual and the further data for the second individual.

10. The method of claim 1 wherein the data on the individual includes facial expressions, physiological information, or accelerometer readings.

11. The method of claim 10 wherein the facial expressions further comprise head gestures.

12. The method of claim 10 wherein the physiological information is collected without physically contacting the individual.

13. The method of claim 1 further comprising inferring cognitive states, based on the data that was collected and the analysis.

14. The method of claim 1 wherein the web server comprises an interface that includes cloud-based storage and a cloud-based server, both remote from the individual.

15. The method of claim 1 wherein the web server comprises an interface that includes datacenter-based storage and a datacenter-based server, both remote to the individual.

16. The method of claim 1 further comprising indexing the data on the individual through the web server.

17. The method of claim 16 wherein the indexing includes categorization based on valence and arousal information.

18. The method of claim 1 further comprising receiving analysis information on a plurality of other individuals, wherein the analysis information allows evaluation of a collective cognitive state of the plurality of other individuals.

19. The method of claim 18 wherein the analysis information includes a correlation for the cognitive state of the plurality of other individuals to the data for the individual that was captured.

20. The method of claim 19 wherein the correlation is based on metadata from the individual and metadata from the plurality of other individuals.

21. The method of claim 1 wherein the analysis which is received from the web server is based on specific access rights.

22. The method of claim 1 further comprising sending a request to the web server for the analysis.

23. (canceled)

24. The method of claim 1 wherein the uploading the data includes only a subset of the data on the individual that was captured.

25-26. (canceled)

27. The method of claim 1 wherein the rendering further comprises recommending a course of action based on the cognitive state of the individual.

28. A computer program product stored on a non-transitory computer-readable medium for distributed analysis, the computer program product comprising code which causes one or more processors to perform operations of:
capturing data for an individual into a computing device, wherein the data provides information for evaluating a cognitive state of the individual;
uploading the data for the individual to a web server;
calculating a cognitive state metric for the individual, on the web server, based on the data that was uploaded;
receiving analysis from the web server, by the computing device, wherein the analysis is based on the data for the individual and the cognitive state metric for the individual; and
rendering an output at the computing device that describes a cognitive state of the individual, based on the analysis that was received.

29. A system for distributed analysis comprising:
a memory which stores instructions;
one or more processors coupled to the memory wherein the one or more processors, when executing the instructions which are stored, are configured to:
capture data for an individual into a computing device, wherein the data provides information for evaluating a cognitive state of the individual;
upload the data for the individual to a web server;
calculate a cognitive state metric for the individual, on the web server, based on the data that was uploaded;
receive analysis from the web server, by the computing device, wherein the analysis is based on the data for the individual and the cognitive state metric for the individual; and
render an output at the computing device that describes a cognitive state of the individual, based on the analysis that was received.
